SEQUENCE LISTING (15 sequences)

SEQ ID NO: 1 - protein, 7 aa, synthetic construct
  REGION 1..7: note = Matrixmetalloprotease (MMP)-responsive sequence
PQGIWGQ

SEQ ID NO: 2 - protein, 8 aa, synthetic construct
  REGION 1..8: note = Matrixmetalloprotease (MMP)-responsive sequence
IPVSLRSG

SEQ ID NO: 3 - protein, 8 aa, synthetic construct
  REGION 1..8: note = Matrixmetalloprotease (MMP)-responsive sequence
VPMSMRGG

SEQ ID NO: 4 - protein, 4 aa, synthetic construct
  REGION 1..4: note = Elastase-responsive sequence
AAPV

SEQ ID NO: 5 - protein, 9 aa, synthetic construct
  REGION 1..9: note = Elastase-responsive sequence
APEEIMDRQ

SEQ ID NO: 6 - protein, 11 aa, synthetic construct
  REGION 1..11: note = Thrombin-responsive sequence
  MOD_RES 4: note = L-Pipecolic Acid
GGFXRYSWGCG

SEQ ID NO: 7 - protein, 10 aa, synthetic construct
  REGION 1..10: note = Thrombin-responsive sequence
  MOD_RES 3: note = L-Cyclohexylalanine
GGXARSWGCG

SEQ ID NO: 8 - protein, 12 aa, synthetic construct
  REGION 1..12: note = FXa-responsive sequence
GGIEGRMGGWCG

SEQ ID NO: 9 - protein, 13 aa, synthetic construct
  REGION 1..13: note = Kalikrein-responsive sequence
CGGGPFRIGGWCG

SEQ ID NO: 10 - protein, 6 aa, synthetic construct
  REGION 1..6: note = Aureolysin-responsive sequence
ADVFEA

SEQ ID NO: 11 - protein, 5 aa, synthetic construct
  REGION 1..5: note = Aureolysin-responsive sequence
AAEAA

SEQ ID NO: 12 - protein, 20 aa, synthetic construct
  REGION 1..20: note = Protease IV-responsive sequence
MKATKLVLGAVILGSTLLAG

SEQ ID NO: 13 - protein, 8 aa, synthetic construct
  REGION 1..8: note = Matrixmetalloprotease (MMP)-responsive sequence
GPQGIAGQ

SEQ ID NO: 14 - protein, 8 aa, synthetic construct
  REGION 1..8: note = Matrixmetalloprotease (MMP)-responsive sequence
GPQGIWGQ

SEQ ID NO: 15 - protein, 15 aa, synthetic construct
  REGION 1..15: note = Matrixmetalloprotease (MMP)-responsive sequence
GCGGPQGIWGQGGCG

(19) United States
(12) Patent Application Publication
(10) Pub. No.: US 2025/0000894 A1
(43) Pub. Date: Jan. 2, 2025

(54) ANTI-INFLAMMATORY VISCOSUPPLEMENTATION AGENT CONTAINING HYALURONIC ACID

(71) Applicant: Bauerfeind AG, Zeulenroda-Triebes (DE)

(72) Inventors: Carsten WERNER, Dresden (DE); Uwe FREUDENBERG, Dresden (DE); Lucas SCHIRMER, Dresden (DE); Beatrix BAUERFEIND-JOHNSON, Zeulenroda-Triebes (DE); Jürgen DIETRICH, Schleiden (DE)

(21) Appl. No.: 18/709,645

(22) PCT Filed: Nov. 15, 2022
(86) PCT No.: PCT/EP2022/081958

§ 371 Date: May 13, 2024

(30) Foreign Application Priority Data
Nov. 15, 2021 (DE) 102021212812.2

(51) Int. Cl. A61K 31/728; A61K 9/00; A61K 9/16; A61P 19/02

(52) U.S. Cl. CPC A61K 31/728; A61K 9/0019; A61K 9/1641; A61K 9/1652; A61K 9/1682; A61P 19/02

(57) ABSTRACT

The present invention relates to therapeutic compositions containing hydrogel particles, at least one hyaluronic acid component and at least one pharmaceutically acceptable excipient as well as methods for producing therapeutic compositions and the use thereof for treating arthritis.

The present invention elates to therapeutic compositions containing hydrogel particles, at least one hyaluronic acid component and at least one pharmaceutically acceptable excipient as well as methods for producing therapeutic compositions and the use thereof for treating arthritis.

Osteoarthritis and rheumatoid arthritis are the most frequent manifestations of arthritis. Osteoarthritis (also referred to as arthrosis) is a chronic-degenerative joint change with cartilage degradation, which is associated with pain and functional limitations. The synovial fluid present in the joint space protects the articular cartilage against mechanical stress. Changes or loss of the synovial fluid can lead to cartilage damage in the joint and osteoarthritis (OA). OA can intensify the degradation of the articular surface and of the synovial fluid (Berenbaum, Osteoarthritis and Cartilage, (2013), doi: 10.1016/j.joca.2012.11.012). The inflammatory joint disease rheumatoid arthritis (chronic polyarthritis, primary chronic polyarthritis, RA), in contrast, is a chronic autoimmune disease. Currently, treatment options are the injection of hyaluronic acid preparations as viscosupplementation therapy to improve the mechanical properties of the synovial fluid and the systemic administration of non-steroidal anti-rheumatic drugs (NSAID) or cortisol. The injection of hyaluronic acid preparations can lead to the temporary improvement of the mobility and reduction of pain, but the inflammation is not treated causatively. Anti-inflammatory medicaments treat the inflammation, but cause side effects. Inflammatory processes in the joint are associated with the infiltration of immune cells, which are stimulated by chemokines, such as IL-8 (CXCL8) and RANTES (CLL5) in the synovial fluid (Vergunst et al., (2005) Scandinavian Journal of Rheumatology, doi: 10.1080/03009740500439159, Valcamonica et al., Clin. Exp., Rheumatol. (2014), Goldring & Berenbaum, Curr. Opin. Pharmacol. 22, 51-63 (2015), Pierzchala et al., Arch. Immunol. Ther. Exp. (Warsz). (2011) doi: 10.1007/s00005-011-0115-4).

The technical problem, on which the present invention is based is to overcome the disadvantages of known methods for the arthritis therapy, in particular OA and RA, and of therapeutic compositions used therein. It is in particular the technical problem of the present invention to provide a therapeutic composition, which leads to an improved treatment of arthritis, which is in particular able to symptomatically and causatively therapize arthritis.

The present invention solves the technical problem on which it is based in particular by means of the subject matter of the independent claims as well as the teachings of the dependent claims and of the present description.

The invention relates to a therapeutic composition containing hydrogel particles, at least one hyaluronic acid component and at least one pharmaceutically acceptable excipient, in particular an aqueous fluid, wherein the hydrogel particles have at least one polyionic polymer component, which is covalently connected to at least one uncharged polymer component and/or, in particular or, a non-polymer crosslinker component by forming a network, and wherein the at least one polyionic polymer component has sulfate or sulfonate groups. These sulfate or sulfonate groups are present in the hydrogel, in particular the hydrogel particles, as free, thus unbonded and ionizable groups, which are in particular negatively charged under physiological conditions. These sulfate or sulfonate groups are also present in the composition according to the invention, thus in the presence of the hyaluronic acid component, as free, thus unbonded and ionizable groups, in particular negatively bonded under physiological conditions.

The invention thus relates to an, in particular anti-inflammatory, composition, in particular for the use as biolubricant, that is, as lubricant for biological tissues, in particular in vivo, which comprises at least three components. These three components are at least one, in particular one, hyaluronic acid component, hydrogel particles as well as at least one pharmaceutically acceptable excipient. The hydrogel particles according to the invention comprise at least two components, namely at least one, in particular one, polyionic polymer component, in particular a polyanionic polymer component, in particular glycosaminoglycan (GAG) component and at least one non-polymer crosslinker component or, optionally and, at least one, in particular one, uncharged polymer component, in particular a polyethylene glycol (PEG) component. The at least three components provided according to the invention are present in the composition according to the invention in mixed form without forming a chemical bond with one another, the hydrogel particles are in particular not chemically covalently connected to the hyaluronic acid component but the hydrogel particles are only present in the pharmaceutically acceptable excipient, in particular aqueous fluid, in physical mixture with the hyaluronic acid component. According to the invention, the hyaluronic acid component is thus present in free, unbonded, only physically associated form with the hydrogel particles and neither the polyionic polymer component nor the uncharged polymer component nor the non-polymer crosslinker component of the hydrogel particles are chemically covalently connected to the hyaluronic acid component, which is present in free form.

According to the invention, the at least one uncharged polymer component is covalently connected to the at least one polyionic polymer component, in particular directly or by means of a crosslinker component, in one embodiment. Together, the uncharged polymer component and the polyionic polymer component covalently connected thereto, thus form the hydrogel particles, optionally in additional presence of the non-polymer crosslinker component, by forming a network.

According to the invention, the at least one non-polymer crosslinker component is covalently connected, in particular directly, to the at least one polyionic polymer component in a further embodiment. Together, the at least one non-polymer crosslinker component and the polyionic polymer component covalently connected thereto, thus form the hydrogel particles by forming a network.

The invention thus provides in particular that the at least one polyionic polymer component is covalently connected directly or via at least one crosslinker component to the at least one uncharged polymer component by forming a network or, in a further embodiment, that the at least one polyionic polymer component is connected to the at least one crosslinker component by forming a network.

According to the invention, the polyionic polymer component has sulfate or sulfonate groups, in particular sulfate groups. According to the invention, these sulfate or sulfonate groups are not covalently connected to the uncharged polymer component, in particular not to the polyethylene glycol component, or the hyaluronic acid component. According to the invention, these sulfate or sulfonate groups are not covalently connected to the non-polymer crosslinker component.

Without being bound to the theory, the sulfate or sulfonate groups are present in the hydrogel essentially in deprotonated form. These deprotonated sulfate or sulfonate groups can experience charge compensations or form charge interactions by means of counter ions and/or proteins. Sulfate or sulfonate groups can thus form reversible bonds to soluble molecules via non-covalent interactions, in particular charge interactions, in particular as soon as they are in contact with biofluids, which preferably have soluble molecules.

In addition to the sulfate or sulfonate groups, the polyionic polymer used for the production of the hydrogel particles preferably has at least two further functional groups, in particular selected from the group consisting of amino group, thiol group, maleimide group, vinylsulfone group, acrylate group, carboxyl group and combinations thereof, with which the polyionic polymer is covalently connected to the uncharged polymer used for the production of the hydrogel particles and/or the non-polymer crosslinker component, so that a hydrogel forms, which forms at least one polyionic polymer component, which is crosslinked via at least one uncharged polymer component or at least one non-polymer crosslinker component or both and thus forms a network. The at least two functional groups of the polyionic polymer can be identical or different, they are in particular identical.

In preferred embodiment of the present invention, the uncharged polymer used to produce the hydrogel particles has, on the respective free end of the polymer, a functional group, in particular end group, selected from the group consisting of amino group, thiol group, maleimide group, vinylsulfone group, acrylate group, carboxyl group and combinations thereof. The uncharged polymer is thus functionalized, in particular end group functionalized. Via these reactive functional groups, in particular end groups, the at least one uncharged polymer is preferably connected to the at least one polyionic polymer and thus forms the network of the hydrogel particles. The at least two functional groups of the uncharged polymer can be identical or different, they are in particular identical.

The uncharged polymer can be linear or branched, in particular multi-armed, that is, in preferred embodiment of the present invention, it consists of several branched chains. The uncharged polymer is in particular star-shaped, that is, multi-armed with a center, from which several, in particular four or eight, in particular four, in particular equally long, chains of corresponding repeating units branch off.

In preferred embodiment of the present invention, the non-polymer crosslinker molecule, which is used to produce the hydrogel particles in preferred embodiments, has at least two functional groups, in particular end groups, selected from the group consisting of amino group, thiol group, maleimide group, vinylsulfone group, acrylate group, carboxyl group, hydroxylated aromatic group and combinations thereof. The non-polymer crosslinker molecule is thus functionalized, in particular end group functionalized. Via these reactive functional groups, in particular end groups, the at least one non-polymer crosslinker molecule is preferably connected to the at least one polyionic polymer and thus forms the network of the hydrogel particles. The at least two functional groups of the non-polymer crosslinker molecule can be identical or different, they are in particular identical.

The therapeutic composition of the present invention is characterized in particular by a particularly advantageous usability for treating arthritis, in particular rheumatoid arthritis, osteoarthritis, infectious arthritis, post-infectious arthritis, psoriatic arthritis or gouty arthritis. The therapeutic composition according to the invention can advantageously in particular bond pro-inflammatory cytokines, in particular chemokines, such as interleukin-8 (IL-8) from the synovial fluid and can thus prevent chemokine-induced immune cell migration. It furthermore has a particularly advantageous viscosupplementation effect, that is, it provides for a reduction of the frictional resistance in the synovial fluid and thus the function of improving the slippage in the joint, here also referred to as lubrication effect, which is preferably at least the same as it as is given in known compositions used in this context, such as hyaluronic acid, but which is in particular better. The lubrication effect provided according to the invention leads to a pain relief in the patient, prevents or reduces functional limitations and restrictions of movement, thus prevents protective postures and counteracts further joint damages. To the inventors' surprise, they determined that the present therapeutic viscosuppelentation composition is characterized in particular by a functional combination of advantageous lubrication effect and the ability to bond, thus sequester, proinflammatory chemokines and thus anti-inflammatory effect, wherein a particularly good injectability of the composition is furthermore at hand. The hyaluronic acid advantageously does not prevent the sequestering effect of the hydrogel particles but leads to surprising advantages. It is particularly advantageous that the sequestering effect of the hydrogel by bonding the proinflammatory cytokines does not only have the result that the inflammatory processes in the tissue are reduced and prevented but that the hyaluronic acid component of the composition is furthermore also protected against degradation, that is, it is degraded more slowly and thus develops its lubrication effect for a longer period of time. The provided combination of anti-inflammatory and improved mechanical properties with respect to the, in particular, extended lubrication effect and good injectability allows a particularly effective intra-articular and thus systematic therapy of arthritis, in particular at least of one of the clinical symptoms thereof, in particular the symptoms thereof, in particular of the entire symptomatology thereof, in a synergistic way.

The therapeutic composition according to the invention containing hydrogel particles, at least one hyaluronic acid component and at least one pharmaceutically acceptable excipient is produced in an embodiment, which is preferred according to the invention, in that at least one uncharged polymer is covalently connected directly or via at least one crosslinker component toat least one polyionic polymer and thereby form a hydrogel, wherein this hydrogel accordingly has at least one uncharged polymer component, which is covalently connected to at least one polyionic polymer component by forming a network, and wherein this hydrogel, in particular in the form of hydrogel particles, is mixed with at least one hyaluronic acid component and at least one pharmaceutically acceptable excipient.

The therapeutic composition according to the invention containing hydrogel particles, at least one hyaluronic acid component and at least one pharmaceutically acceptable excipient is produced in an embodiment, which is preferred according to the invention, in that at least one non-polymer crosslinker molecule is covalently connected directly to at least one polyionic polymer and thereby form a hydrogel, wherein this hydrogel accordingly has at least one non-polymer crosslinker component, which is covalently connected to at least one polyionic polymer component by forming a network, and wherein this hydrogel, in particular in the form of hydrogel particles, is mixed with at least one hyaluronic acid component and at least one pharmaceutically acceptable excipient.

In a preferred embodiment, the at least one uncharged polymer has an average molecular weight of 5,000 to 25,000, in particular 10,000 to 19.000 Da.

In a preferred embodiment, the repeating unit of the at least one uncharged polymer component has an average molecular weight of 30 to 55, in particular of 40 to 50 Da auf.

In a particularly preferred embodiment, the at least one uncharged polymer component is a linear uncharged polymer component.

In a particularly preferred embodiment, the at least one uncharged polymer component is a branched, in particular multi-arm, uncharged polymer component.

In a particularly preferred embodiment, the at least one multi-arm uncharged polymer component is a four-arm or eight-arm uncharged polymer component.

In a particularly preferred embodiment, the at least one uncharged polymer component is selected from the group consisting of polyethylene glycol (PEG) component, poly(2-oxazoline) (POX) component, polyvinylpyrrolidone (PVP) component, polyvinyl alcohol (PVA) component, polyacrylamide (PAM) component and combinations thereof.

In a particularly preferred embodiment, the at least one multi-arm uncharged polymer component is a PEG component.

In a particularly preferred embodiment, the at least one uncharged polymer component is a polyethylene glycol component, in particular a linear or multi-arm polyethylene glycol component.

In a particularly preferred embodiment, the at least one multi-arm uncharged polymer component is a four-arm polyethylene glycol component.

In a further preferred embodiment, the at least one multi-arm uncharged polymer component is an eight-arm polyethylene glycol component.

In a particularly preferred embodiment, the at least one multi-arm uncharged polymer component is star PEG, likewise known as starPEG.

In a preferred embodiment, the uncharged polymer used for the production of the hydrogel particles according to the invention has, on the respective free end of the polymer chain, a functional group, in particular end group, selected from the group consisting of amino group, thiol group, maleimide group, vinyl sulfone group, acrylate group, carboxyl group and combinations thereof. The uncharged polymer is thus in particular end group-functionalized. The at least two functional groups of the uncharged polymer can be identical or different, they are in particular identical.

In a particularly preferred embodiment, the uncharged polymer used for the production of the hydrogel particles according to the invention has at least two amino groups, in particular terminal amino groups.

In a particularly preferred embodiment, the uncharged polymer used for the production of the hydrogel particles according to the invention has at least two carboxyl groups, in particular terminal carboxyl groups.

In a preferred embodiment, the polyionic polymer component is a polyanionic polymer component, in particular a glycosaminoglycan component.

In a particularly preferred embodiment, the glycosaminoglycan used for the production of the hydrogel particles according to the invention is selected from the group consisting of chondroitin sulfate, dextran sulfate, dermatan sulfate, glucosamine sulfate, heparin, selectively desulfated heparin, heparan sulfate and hyaluronan sulfate. The glycosaminoglycan is in particular heparin, heparan sulfate or dextran sulfate, in particular heparin or selectively desulfated heparin, in particular selectively N (nitrogen)-desulfated heparin.

In a particularly preferred embodiment, the glycosaminoglycan used for the production of the hydrogel particles according to the invention is a desulfated glycosaminoglycan, in particular a selectively N-desulfated glycosaminoglycan, in particular a selectively N-desulfated heparin.

In a particularly preferred embodiment, the glycosaminoglycan used for the production of the hydrogel particles according to the invention is a selectively N-desulfated heparin, which can be obtained according to the synthesis rule from Atallah et. al. (Biomaterials. 2018 October; 181:227-239.doi: 10.1016/j.biomaterials.2018.07.056. Epub 2018 Jul. 30).

In a preferred embodiment, the polyionic polymer component used for the production of the hydrogel particles according to the invention is poly(4-styrenesulfonic acid-co-maleic acid).

In a preferred embodiment, the average molecular weight of the polyionic polymer is 3 to 20, in particular 4 to 14 kDa, in particular 10 to 14 kDa, in particular 13 to 14 kDa.

In a preferred embodiment, 40 to 80, in particular 40 to 75, in particular 40 to 70, in particular 65 to 80, in particular 65 to 75 sulfate or sulfonate groups are present for each polyionic polymer molecule.

In a preferred embodiment, 40 to 55, in particular 40 to 50, in particular 45 to 50 sulfate or sulfonate groups are present for each desulfated glycosaminoglycan, in particular for each selectively N-desulfated glycosaminoglycan, in particular for each selectively N-desulfated heparin.

In a preferred embodiment, the repeating unit of the at least one polyionic polymer component has an average molecular weight of 400 to 550, in particular of 420 to 470 Da.

In a preferred embodiment, the repeating unit of the at least one polyionic polymer component has an average molecular weight of 400 to 550, in particular of 450 to 550, in particular 500 to 550 Da.

In a preferred embodiment, 1.0 to 4.5, in particular 1.5 to 4.5, in particular 2.0 to 3.0 sulfate or sulfonate groups are present for each repeating unit of the at least one polyionic polymer component.

In a preferred embodiment, 1.0 to 4.5, in particular 1.5 to 2.0 sulfate or sulfonate groups are present for each repeating unit of the at least one polyionic polymer component, in particular for each desulfated glycosaminoglycan, in particular for each selectively N-desulfated glycosaminoglycan, in particular for each selectively N-desulfated heparin.

In a preferred embodiment, the non-polymer crosslinker molecule used for the production of the hydrogel particles according to the invention, in preferred embodiment of the present invention, has at least two functional groups, which are suitable for forming a respective covalent bond to the polyionic polymer component, in particular selected from the group consisting of amino group, thiol group, maleimide group, vinylsulfone group, acrylate group, carboxyl group, hydroxylated aromatic group and combinations thereof.

The non-polymer crosslinker molecule representing the non-polymer crosslinker component in the hydrogel is thus functionalized, in particular end group functionalized. The at least one non-polymer crosslinker molecule is preferably connected to the at least one polyionic polymer via these reactive functional groups and thus forms the network of the hydrogel particles. The at least two functional groups of the non-polymer crosslinker molecule polymer can be identical or different, they are in particular identical.

In a preferred embodiment, the crosslinker molecule is a non-polymer bifunctional crosslinker molecule.

In a preferred embodiment, the crosslinker molecule has at least two amino groups. In a particularly preferred embodiment, the crosslinker molecule with at least two amino groups is selected from the group consisting of ethylene diamine, propylene diamine (1,3-diaminopropane), butan-1,4-diamine, pentane-1,5-diamine (cadaverine), hexamethylene-1,6-diamine and combinations thereof.

In a preferred embodiment, the crosslinker molecule has at least two carboxyl groups. In a particularly preferred embodiment, the crosslinker molecule with at least two carboxyl groups is selected from the group consisting of oxalic acid, malonic acid, succinic acid, glutaric acid, adipic acid, pimelic acid and combinations thereof.

In a particularly preferred embodiment, the crosslinker molecule is a molecule with at least two different functional groups, in particular at least two functional groups, which are able to crosslink the polyionic polymer component and the uncharged polymer component, in particular N-(2-aminoethyl) maleimide.

In a preferred embodiment, the at least one non-polymer crosslinker component is selected from the group consisting of ethylene diamine, propylene diamine (1,3-diaminopropane), butane-1,4-diamine, pentane-1,5-diamine (cadaverine), hexamethylene-1,6-diamine oxalic acid, malonic acid, succinic acid, glutaric acid, adipic acid, pimelic acid, N-(2-aminoethyl) maleimide and combinations thereof.

In particularly preferred embodiment, the crosslinker molecule is in particular an enzymatically cleavable sequence, in particular a matrix metalloproteases (MMPs)-responsive element, for example PQGIWGQ, IPVSLRSG or VPMSMRGG, cathepsin-responsive element, such as VPMSMRGG, elastase-responsive element, for example AAPV or APEEIMDRQ, anti-blood clotting enzyme-responsive element, for example thrombin-responsive GGF-pipecolic acid-RYSWGCG or GG-cyclohexylalanine-ARSWGCG, FXa-responsive element, for example GGIEGRMGGWCG, kallikrein-responsive elements, for example CGGGPFRIGGWCG or bacterial proteases-responsive element, for example aureolysin-responsive ADVFEA or AAEAA, elastase-responsive element, such as AAPV or the protease IV-responsive sequence MKATKLVLGAVILGSTLLAG, in particular with one of the sequences GPQGIAGQ, GPQGIWGQ or GCGGPQGIWGQGGCG. The sequences GPQGIAGQ and GPQGIWGQ are artificially generated sequences, which are cleavable by means of a plurality of matrix-metalloproteases (MMPs), in particular MMP1, 3, 7, 9.

In a preferred embodiment, the enzymatically cleavable sequences are each flanked at the C- and N-terminal end by a cysteine, in particular of the sequence GCG or GCGG. The polyionic polymer and the uncharged polymer or at least two polyionic polymers is crosslinked via the respective cysteine.

In a preferred embodiment, the polyionic polymer component preferably used for the production of the hydrogel particles according to the invention is covalently linked to a non-polymer crosslinker component, in particular peptide.

In a preferred embodiment, the non-polymer crosslinker component used for the production of the hydrogel particles according to the invention and the polyionic polymer component are covalently linked to one another by means of a non-polymer crosslinker component, in particular peptides.

In a preferred embodiment, the uncharged polymer component and the polyionic polymer component are covalently connected to one another via at least one amide bond.

In a preferred embodiment, the uncharged polymer component and the polyionic polymer component are covalently connected to one another by means of a non-polymer crosslinker component via at least one amide bond.

In a further preferred embodiment, the uncharged polymer component used for the production of the hydrogel particles according to the invention and the polyionic polymer component are covalently connected directly to one another, in particular by means of an amide bond, thiol-amine bond, disulfide bond or bio-orthogonal thioether bond, which can be obtained from thiol-maleimide, thiol-vinylsulfone or thiol-acrylate reaction.

In a preferred embodiment, the uncharged polymer component used for the production of the hydrogel particles according to the invention, in particular uncharged polymer component having amino groups, and the polyionic polymer component, in particular polyionic polymer component having carboxyl groups, are covalently connected directly to one another by means of an amide bond. In a preferred embodiment, the amide bond is made possible by means of 1-ethyl-3-(3-dimethylaminopropyl) carbodiimide/N-hydroxysulfosuccinimide (EDC/sNHS)) activation at the carboxyl groups of the polyionic polymer component.

In a further preferred embodiment, the non-polymer crosslinker component used for the production of the hydrogel particles according to the invention, and the polyionic polymer component are covalently connected directly to one another, in particular by means of an amide bond, thiol-amine-bond, disulfide bond or bio-orthogonal thioether bond, which can be obtained from thiol-maleimide, thiol-vinylsulfone or thiol-acrylate reaction.

In a preferred embodiment, the non-polymer crosslinker component used for the production of the hydrogel particles according to the invention, in particular non-polymer crosslinker component having amino groups, and the polyionic polymer component, in particular polyoionic polymer component having carboxyl groups, are covalently connected directly to one another by means of an amide bond. In a preferred embodiment, the amide bond is made possible by means of 1-ethyl-3-(3-dimethylaminopropyl) carbodiimide/N-hydroxysulfosuccinimide (EDC/sNHS) activation at the carboxyl groups of the polyionic polymer component.

In a preferred embodiment, the hydrogel particles have at least two, in particular two, different non-polymer crosslinker components. In a particularly preferred embodiment, the hydrogel particles have a non-polymer crosslinker component, which has at least two carboxyl groups, is in particular end group functionalized with a respective carboxyl group, and a non-polymer crosslinker component, which has at least two amino groups, is in particular end group functionalized with a respective amino group. The non-polymer crosslinker component having at least two carboxyl groups can preferably be connected via at least one amide bond to the non-polymer crosslinker component preferably having amino groups and the non-polymer crosslinker component having at least two amino groups can be connected via at least one amide bond to the polyionic polymer component, in particular heparin, preferably having carboxyl groups, wherein in a preferred embodiment, the amide bond is made possible by means of 1-ethyl-3-(3-dimethylaminopropyl) carbodiimide/N-hydroxysulfosuccinimide (EDC/sNHS) activation at the carboxylic groups of the non-polymer crosslinker molecule and the polyionic polymer component.

In a preferred embodiment, the hydrogel particles have at least two, in particular two, different uncharged polymer components. In a particularly preferred embodiment, the hydrogel particles have an uncharged polymer component, which has at least two carboxyl groups, is in particular end group-functionalized with a respectively carboxyl group, and an uncharged polymer component, which has at least two amino groups, is in particular end group functionalized with a respective amino group. The uncharged polymer component having at least two carboxyl groups can preferably be connected via at least one amide bond to the uncharged polymer component preferably having amino groups and the uncharged polymer component having at least two amino groups can be connected via at least one amide bond to the polyionic polymer component, in particular heparin, preferably having carboxyl groups, wherein in a preferred embodiment, the amide bond is made possible by means of 1-ethyl-3-(3-dimethylaminopropyl) carbodiimide/N-hydroxysulfosuccinimide (EDC/sNHS) activation at the carboxyl groups of the uncharged polymer component and the polyionic polymer component.

In a preferred embodiment, the hydrogel particles have an uncharged polymer component. In a particularly preferred embodiment, the hydrogel particles have an uncharged polymer component, which has at least two amino groups, is in particular end group functionalized with a respective amino group. The uncharged polymer component having at least two amino groups can preferably be connected via at least one amide bond to the polyionic polymer component, in particular heparin, preferably having carboxyl groups, wherein in a preferred embodiment, the amide bond is made possible by means of 1-ethyl-3-(3-dimethylaminopropyl) carbodiimide/N-hydroxysulfosuccinimide (EDC/sNHS) activation at the carboxyl groups of the polyionic polymer component.

In a particularly preferred embodiment, the uncharged polymer, which is in particular end group functionalized with a respective carboxyl group, is a multi-arm, in particular eight-arm, PEG.

In a particularly preferred embodiment, the uncharged polymer, which is end group functionalized in particular with a respective amino group, is a multi-arm, in particular four-arm, PEG.

In a particularly preferred embodiment, the hydrogel particles are present in the therapeutic composition in a swollen state, in particular in aqueous solution, in particular water, or buffered aqueous solution, in particular PBS, swollen form.

In a preferred embodiment, the hydrogel particles can swell up to 0.75- to 2.5-times the size of the hydrogel particles directly after the hydrogel formation.

In a preferred embodiment, the content of the hydrogel particles in swollen state in the composition according to the invention is 5 to 60% by volume, in particular 9 to 55% by volume, in particular 10 to 50% by volume (in each case based on the total volume of the composition).

In a preferred embodiment, the hydrogel particles, in particular in the swollen state, have a storage modulus of maximally 22.0 kPa, in particular maximally 20.0 kPa, in particular maximally 15.0 kPa, in particular maximally 10.0 kPa.

In a preferred embodiment, the hydrogel particles, in particular in the swollen state, have a storage modulus of 0.1 to 22.0 kPa, in particular 0.1 to 20.0 kPa, in particular less than 0.1 to 15.0 kPa, in particular less than 0.1 to 10.0 kPa.

In a preferred embodiment, the hydrogel particles, in particular in the swollen state, have an average size of maximally 200 μm, in particular maximally 80 μm.

In a preferred embodiment, the hydrogel particles, in particular in the swollen state, have an average diameter (size) of 10 to 200 μm, in particular 5 to 150 μm, in particular 10 to 80 μm. The average diameter of the hydrogel particles is preferably determined by means of fluorescence microscopy, preferably with a dye, which has a reactive group, in particular an amine group.

In a preferred embodiment, the hydrogel particles, in particular in the swollen state, have a spherical shape.

In a preferred embodiment, the hydrogel particles, in particular in the swollen state, have a size distribution with a deviation of less than 15%, in particular less than 10%, of the average size.

In a preferred embodiment, the hydrogel particles, in particular in the swollen state, have a sulfate or sulfonate group concentration of at least 0.1 mmol/l, in particular at least 10 mmol/l, in particular at least 100 mmol/l, in particular at least 0.1 to 800 mmol/l, in particular 10 to 800 mmol/l, in particular 20 to 500 mmol/l, in particular 20 to 200 mmol/l, in particular 50 to 200 mmol/l, in the volume of the hydrogel particles.

In a particularly preferred embodiment, the hydrogel particles have sulfate groups. In a particularly preferred embodiment, the hydrogel particles, in particular in the swollen state, have sulfate groups in a concentration of at least 0.1 mmol/l, in particular at least 10 mmol/l, in particular at least 100 mmol/l, in particular 0.1 to 800 mmol/l, in particular 10 to 800 mmol/l, in particular 20 to 500 mmol/l, in particular 20 to 200 mmol/l, in particular 50 to 200 mmol/l, in the volume of the hydrogel particles, which are present in the swollen state.

In a particularly preferred embodiment, the hydrogel particles have sulfonate groups. In a particularly preferred embodiment, the hydrogel particles, in particular in the swollen state, have sulfonate groups in a concentration of at least 0.1 mmol/l, in particular at least 10 mmol/l, in particular at least 100 mmol/l, in particular 0.1 to 800 mmol/l, in particular 10 to 800 mmol/l, in particular 20 to 500 mmol/l, in particular 20 to 200 mmol/l, in particular 50 to 200 mmol/l, in the volume of the hydrogel particles, which are present in the swollen state.

In a preferred embodiment, the hydrogel particles have an average mesh width of at least 5 nm, in particular at least 5.7 nm, auf. The minimum average mesh width ensures that all relevant signal molecules with a diameter of 3 to 5 nm can penetrate the hydrogel particles quickly and sterically, and the ability of the signal molecules of diffusing through the particles is essentially a function of the charge properties of the particles and the interaction with the signal molecules resulting therefrom.

In a preferred embodiment, the hydrogel particles have an average mesh width of 5 to 30 nm, in particular 5.7 to 30 nm.

In a preferred embodiment, the composition according to the invention has a content of 0.1 to 3.0% by weight, in particular 0.4 to 2.0% by weight, in particular 0.5 to 2.0% by weight, in particular 0.5 to 1.8% by weight of hyaluronic acid component (in each case based on the total mass of the composition).

In a preferred embodiment, the composition according to the invention has a content of 5 to 20% by volume, in particular 10 to 20% by volume. in particular 10 to 18% by volume, of hyaluronic acid component (in each case based on the total volume of the composition), in particular a hyaluronic acid solution with 10% by weight (based on the total mass of the hyaluronic acid solution).

In a preferred embodiment, the hyaluronic acid component is hyaluronic acid and/or a salt thereof, the hyaluronic acid component is in particular the sodium salt of the hyaluronic acid.

In a preferred embodiment, the hyaluronic acid component is hyaluronic acid with an average molecular weight of 4 to 100 000 kDa, in particular 500 to 100 000 kDa, in particular 1 000 to 7 000 kDa, in particular 1 000 to 5 000 kDa.

In a preferred embodiment, the composition according to the invention has a content of 30 to 90% by volume, in particular 30 to 85% by volume, in particular 40 to 80% by volume, of pharmaceutically acceptable excipient (in each case based on the total volume of the composition).

In a particularly preferred embodiment, the at least one pharmaceutically acceptable excipient is an aqueous fluid, in particular water or an aqueous buffered solution, in particular phosphate-buffered saline solution (PBS).

In a particularly preferred embodiment, the at least one pharmaceutically acceptable excipient, in particular the aqueous fluid, has a pH value of 6.5 to 8.0, in particular 6.8 to 7.6, in particular 7.0 to 7.5, in particular 7.4.

In a particularly preferred embodiment, the therapeutic composition additionally has at least one additive, in particular marine collagen, sorbitol, mannitol, platelet-rich plasma (PRP), polyphenols, S-allyl-cysteine, pentosan-polyphosphate-Na, and/or extracellular vesicles, containing curcuminoids, in particular sorbitol.

In a preferred embodiment, the solids content of the composition according to the invention is 10 to 25% by weight, in particular 10 to 17% by weight, in particular 12 to 15% by weight (in each case based on the total mass of the composition).

In a preferred embodiment, the composition according to the invention has a viscosity of 10 to 100, in particular 20 to 90, in particular 30 to 80 Pa/s.

In a preferred embodiment, the composition according to the invention is an injectable therapeutic composition.

In a preferred embodiment, the injection force for injecting the composition according to the invention at an injection speed of 0.05 ml/s through a 25 G needle is maximally 10 N, in particular 2.5 to 3.5 N, in particular 2.7 to 3.5 N. In a particularly preferred embodiment, the hydrogel particles are present in the swollen state when measuring the injection force.

In a preferred embodiment, the friction coefficient of the composition according to the invention is maximally 0.2, in particular 0.085 to 0.095, in each case at 10 revolutions/s.

In a preferred embodiment, the composition according to the invention is able to reduce the concentration of at least one free cytokine, in particular chemokine, in particular IL-8, in a surrounding solution, in particular a synovial fluid. In preferred embodiment, the composition according to the invention has the ability to reduce the concentration of at least one free cytokine by 70 to 80% in a cytokine-containing model synovial fluid.

In a particularly preferred embodiment of the present invention, the free cytokine, in particular chemokine, is a pro-inflammatory cytokine, in particular chemokine.

In a particularly preferred embodiment, the free cytokine is selected from the group consisting of IL-8, IP-10, MCP-1, MIP-1alpha, MIP-1beta, RANTES and combinations thereof, in particular IL-8.

The present invention also relates to therapeutic compositions for use in a therapeutic method for treating arthritis, in particular osteoarthritis, rheumatoid arthritis, infectious arthritis, post-infectious arthritis, psoriatic arthritis or gouty arthritis, in particular for the application, in particular injection, in particular intra-articular injection, into a joint of a human or animal patient.

The present invention also relates to the use of the hydrogel particles used in the present therapeutic composition in a therapeutic method for treating arthritis, in particular osteoarthritis, rheumatoid arthritis, infectious arthritis, post-infectious arthritis, psoriatic arthritis or gouty arthritis, in particular together with at least one hyaluronic acid component and at least one pharmaceutically acceptable excipient as well as optionally at least one additive.

The invention in particular relates to therapeutic methods for treating arthritis, in particular osteoarthritis, rheumatoid arthritis, infectious arthritis, post-infectious arthritis, psoriatic arthritis or gouty arthritis, by using a therapeutic composition of the present invention.

The present invention also relates to a method for the therapy of arthritis, in particular osteoarthritis, rheumatoid arthritis, infectious arthritis, post-infectious arthritis, psoriatic arthritis or gouty arthritis, wherein a therapeutic composition of the present invention is applied to, in particular injected into, a human or animal patient in an effective quantity, in particular into a joint, thus injected intra-articularly.

The invention relates in particular to methods for the therapy of arthritis, in particular osteoarthritis, rheumatoid arthritis, infectious arthritis, post-infectious arthritis, psoriatic arthritis or gouty arthritis, by using a therapeutic composition, in particular containing hydrogel particles, at least one hyaluronic acid component and a pharmaceutically acceptable excipient, wherein the hydrogel particles have at least one polyethylene glycol component, which is covalently connected to at least one polyionic polymer component, and wherein the at least one polyionic polymer component has sulfate or sulfonate groups and wherein an effective quantity of the composition according to the invention is applied to, in particular injected intra-articularly into, a human or animal patient.

In a particularly preferred embodiment of the present invention, the therapeutic composition and the therapeutic method for treating arthritis is characterized in that the therapy of arthritis is achieved by means of the lubrication effect, which is provided according to the invention, the cytokine-reducing effect, which is provided according to the invention, in particular anti-inflammatory effect, or both.

The present invention also relates to therapeutic compositions of the present invention for use in a therapeutic method for treating lower back pain, in particular for the application, in particular injection of a human or animal patient.

The present invention also relates to the use of the hydrogel particles used in the present therapeutic composition in a therapeutic method for treating lower back pain, in particular together with at least one hyaluronic acid component and at least one pharmaceutically acceptable excipient as well as optionally at least one additive.

The invention relates in particular to therapeutic methods for treating lower back pain by using a therapeutic composition of the present invention.

The present invention also relates to a method for the therapy of lower back pain, wherein a therapeutic composition of the present invention is applied to, in particular injected into a human or animal patient.

The invention relates in particular to methods for the therapy of lower back pain by using a therapeutic composition, in particular containing hydrogel particles, at least one hyaluronic acid component and a pharmaceutically acceptable excipient, wherein the hydrogel particles have at least one polyethylene glycol component, which is covalently connected to at least one polyionic polymer component, and wherein the at least one polyionic polymer component has sulfate or sulfonate groups and wherein an effective quantity of the composition according to the invention is applied to, in particular injected into a human or animal patient.

The present invention also relates to methods for producing a therapeutic composition, in particular of the present invention, comprising the following method steps:
a) providing at least one uncharged polymer, in particular a functionalized uncharged polymer, and/or at least one non-polymer functionalized crosslinker molecule, and at least one polyionic polymer having sulfate or sulfonate groups, at least one hyaluronic acid component and at least one pharmaceutically acceptable excipient as well as optionally further components,
b) crosslinking the uncharged polymer and/or non-polymer crosslinker molecule with the polyionic polymer by forming a network for obtaining a hydrogel, in particular of hydrogel particles, from at least one polyionic polymer component, which is covalently connected to at least one uncharged polymer component and/or a non-polymer crosslinker component,
c) mixing the hydrogel, in particular the hydrogel particles, with the at least one hyaluronic acid component and the at least one pharmaceutically acceptable excipient, and
d) obtaining the therapeutic composition.

In preferred embodiment, the, preferably functionalized, uncharged polymer has at least two functional groups, in particular end group, selected from the group consisting of amino group, thiol group, maleimide group, vinylsulfone group, acrylate group, carboxyl group and combinations thereof, wherein the at least two groups can be identical or different.

In preferred embodiment, the, preferably functionalized, non-polymer crosslinker molecule has at least two functional groups, in particular end group, selected from the group consisting of amino group, thiol group, maleimide group, vinylsulfone group, acrylate group, carboxyl group hydroxylated aromatic group and combinations thereof, wherein the at least two groups can be identical or different.

In preferred embodiment, the polyionic polymer has, in addition to the at least one sulfate or sulfonate groups, at least two further functional groups, selected from the group consisting of amino group, thiol group, maleimide group, vinylsulfone group, acrylate group, carboxyl group and combinations thereof, wherein the at least two groups can be identical or different.

The respective at least two functional groups selected from the group consisting of amino group, thiol group, maleimide group, vinylsulfone group, acrylate group, carboxyl group and combinations thereof, which are preferably provided in the at least one polyionic polymer and the at least one uncharged polymer and/or non-polymer crosslinker molecule used according to the invention, are in each case selected for the polyionic polymer and the uncharged polymer and/or non-polymer crosslinker molecule, so that the functional groups of the reaction partners can form a covalent bond between the polyionic polymer and the at least one uncharged polymer or between the at least one polyionic polymer and the at least one non-polymer crosslinker molecule by forming a network.

The uncharged polymers are preferably selected from the group consisting of polyethylene glycols (PEG), poly(2-oxazoline) (POX), polyvinylpyrrolidones (PVP), polyvinyl alcohols (PVA) and/or polyacrylamides (PAM), which, in preferred embodiment, have at least two or more functional groups, in particular amino groups, which can be crosslinked with the preferred polyionic polymers having carboxyl groups as functional group.

In particularly preferred embodiment, the carboxyl groups are activated in method step b) by polymer having said carboxyl groups or non-polymer crosslinker molecules 1-ethyl-3-(3-dimethylaminopropyl) carbodiimide/N-hydroxysulfosuccinimide (EDC/sNHS).

In a preferred embodiment, the uncharged polymer is PEG and the polyionic polymer is glycosaminoglycan. In a preferred embodiment, the molar ratio of the polyethylene glycol and of the glycosaminoglycan during the crosslinking in method step b) is 0.75 to 3.0 (PEG/GAG), in particular 0.75, in particular 1.5, in particular 2.25, in particular 3.0.

In a preferred embodiment, the molar ratio of the polyethylene glycol and of the glycosaminoglycan during the crosslinking in method step b) is 0.75 to 3.0 (PEG/GAG), in particular 0.75 with a solids content of 12.0 to 15.0% (m/V), in particular 13.3% (m/V), in particular 1.5 with a solids content of 12.0 to 15.0% (m/V), in particular 13.3% (m/V), in particular 2.25 with a solids content of 12.0 to 15.0% (m/V), in particular 13.3% (m/V), in particular 3.0 with a solids content of 12.0 to 15.0% (m/V), in particular 13.3% (m/V), in each case based on the total volume of the mixture comprising polyethylene glycol and glycosaminoglycan.

In a preferred embodiment, the concentration of polyionic polymer in method step b) is 0.5 to 6.0 mmol/l (based on the mixture of at least one uncharged polymer and at least one polyionic polymer).

In a preferred embodiment, the concentration of uncharged polymer in method step b) is 5.0 to 12.0 mmol/l (based on the mixture of at least one uncharged polymer and at least one polyionic polymer).

In a preferred embodiment, the concentration of non-polymer crosslinker molecule in method step b) is 5.0 to 24.0 mmol/l (based on the mixture of at least one non-polymer crosslinker molecule and at least one polyionic polymer).

In a preferred embodiment, the hydrogel particles are obtained in method step b) by means of mechanical comminution, in particular fragmentation, grinding, shredding, high-pressure methods, in particular ultrasonic treatment and subsequent filtration, in particular filtration by means of a filter device with a pore diameter of maximally 200 μm, from the hydrogel obtained in method step b).

In a preferred embodiment, the hydrogel particles will be produced in method step b) by means of mechanical comminution, in particular fragmentation, grinding, shredding, high-pressure methods, in particular ultrasonic treatment and subsequent filtration, in particular filtration by means of a filter device with a pore diameter of maximally 200 μm, from the hydrogel obtained in method step b), or by means of cryogelation and subsequent lyophilization of the obtained hydrogel particles or by means of microfluidic co-flow gelation methods.

In a particular embodiment of the present invention, the hydrogel particles produced in method step b) are produced so that, after mechanical comminution from the hydrogel obtained in method step b), in particular fragmentation, grinding, shredding, high-pressure methods, in particular ultrasonic treatment and subsequent filtration, or after cryogelation or after microfluidic co-flow gelation methods they have a particulate structure, are in particular present in the form of particles, in particular with a particle size with an average diameter of maximally 200 μm in the swollen state.

In a preferred embodiment, the hydrogel, the hyaluronic acid component and the at least one pharmaceutically acceptable excipient are mixed in method step c) by stirring, in particular at a stirring speed of 500 U/min.

In a particularly preferred embodiment, the hydrogel, the hyaluronic acid component and the pharmaceutically acceptable excipient are mixed in method step c) for 1 to 20 min, in particular 8 to 12, in particular 10 minutes.

In a preferred embodiment, the hydrogel, in particular the hydrogel particles, are swollen prior to the mixing in method step c), in particular in PBS.

In a preferred embodiment, the hydrogel, in particular the hydrogel particles, are concentrated prior to the mixing in method step c), in particular by means of centrifugation, in particular by means of centrifugation for 5 min at 3000G, wherein at least a portion of the supernatant is preferably removed after the centrifugation.

In a preferred embodiment, the invention relates to a method for producing a therapeutic composition, wherein the hydrogel particles are formed in method step b) from the hydrogel obtained in method step b) by means of the following method steps:
b1) fragmentation of the hydrogel, in particular by means of ultrasonic treatment with an ultrasonic homogenizer, to obtain hydrogel fragments and
b2) filtration of the hydrogel fragments to obtain hydrogel particles.

In a particularly preferred embodiment, the hydrogel is swollen prior to the fragmentation according to method step b1), in particular completely, in particular in PBS.

In a particularly preferred embodiment, the fragmentation of the hydrogel is performed in method step b1) by means of grinding, shredding, high-pressure treatment or ultrasonic treatment, in particular ultrasonic treatment.

In a preferred embodiment, the solids content of the hydrogel, obtained in method step b1), is 9 to 13% by weight (based on the total mass of the hydrogel).

In a particularly preferred embodiment, the fragmentation is performed in method step b1) for 1 to 15 minutes.

In a preferred embodiment, the filtration according to method step b2) is performed by means of a filter with a pore diameter in a range of 5 to 200, 10 to 200, in particular 10 to 80, in particular 50 to 200, in particular 110 to 200, in particular 200 μm.

In a preferred embodiment, the invention relates to methods for producing a therapeutic composition, wherein in method step b), the hydrogel, in particular the hydrogel particles, are formed by means of the following method steps:
b1′) producing an emulsion containing the at least one polyionic polymer as well as the at least one uncharged polymer and/or non-polymer crosslinker molecule,
b2′) cooling down the emulsion containing the least one polyionic polymer as well as the at least one uncharged polymer and/or non-polymer crosslinker molecule for obtaining cryogelated hydrogel particles,
b3′) lyophilization of the hydrogel particles,
b4′) purification of the lyophilized hydrogel particles,
wherein the at least one polyionic polymer as well as the at least one uncharged polymer and/or non-polymer crosslinker molecule are crosslinked during the method steps b1′) to b3′) to obtain the hydrogel.

In a preferred embodiment, an emulsion containing the at least one uncharged polymer and the at least one polyionic polymer is produced in toluol in method step b1′).

In a particularly preferred embodiment, the method parameters and those of the cryogelation are adjusted in method steps b1′) to b3′) so that the obtained hydrogel particles have particulate form, are in particular present as particles, in particular with an average particle size (average diameter) in the swollen state of maximally 200 μm.

In a preferred embodiment, the present invention relates to methods for producing a therapeutic composition, wherein hydrogel particles are formed in method step b) by means of the following additional method steps:
b1″) mixing the at least one polyionic polymer as well as the at least one uncharged polymer and/or non-polymer crosslinker molecule,
b2″) forming hydrogel particles by means of a microfluidic device, in particular a microfluidic chip,
b3″) purifying the hydrogel particles, wherein the at least one polyionic polymer as well as the at least one uncharged polymer and/or non-polymer crosslinker molecule are crosslinked during method step b2″) to obtain the hydrogel.

In a preferred embodiment, the microfluidic device has a crossover arrangement, wherein the mixture of polyionic polymer and uncharged polymer and/or non-polymer crosslinker molecule flows through a first crossover input and the first crossover output lying opposite said first crossover input, and an organic phase flows over a second crossover input and the second crossover output lying opposite said second crossover input, wherein the flow direction of the mixture of polyionic polymer and uncharged polymer and/or non-polymer crosslinker molecule and organic phase run perpendicular to one another. According to this embodiment, the microfluidic device preferably has a diameter of the crossover input of 5 to 50 μm.

In a particularly preferred embodiment, the method parameters and the microfluidic device are set so that the obtained hydrogel particles have particulate form, in particular are present as particles, in particular with an average particle size (average diameter) in the swollen state of maximally 200 μm.

In a particularly preferred embodiment, the mixture of charged and polyionic polymer has a flow speed of 5 to 25, in particular 10 to 20 μl/min. In a particularly preferred embodiment, the organic phase has a flow speed of 5 to 25, in particular 10 to 20 μl/min. In a particularly preferred embodiment, the organic phase comprises 3-ethoxy-1,1,1,2,3,4,4,5,5,6,6,6-dodecafluor-2-(trifluoromethyl)-hexane (also known as Novec™ 7500 from 3M) and/or a perfluoropolyether-polyethylene glycol-perfluoropolyether-triblock copolymer (PFPE-PEG-PFPE), in particular a triblock copolymer of Krytox™ 157 FSH (DuPont)-Jeffamine™ ED 600 Amine (Huntsman)-Krytox™ 157 FSH (DuPont), which can be obtained according to the synthesis rule published in Lab Chip, 2008, 8, 1632-1639, in particular 1.8% (m/V).

In a preferred embodiment, the at least one polyionic polymer as well as the at least one uncharged polymer and/or non-polymer crosslinker molecule are mixed in method step b1″) at 2 to 10° C., in particular 2 to 8° C., in particular 2 to 6° C.

In a preferred embodiment, the hydrogel particles formed in method step b2′) are stored in a further method step b2a″) prior to the purification in method step b3′), in particular for up to 14 hours.

In a preferred embodiment, the hydrogel particles are first separated in method step b3″) by means of a mixture comprising 1H,1H,2H,2H-perfluoro-1-octanol and 3-ethoxy-1,1,1,2,3,4,4,5,5,6,6,6-dodecafluor-2-(trifluormethyl)-hexane, in particular a mixture comprising a volume ratio of 1:1 of 1H, 1H,2H,2H-perfluoro-1-octanol and 3-ethoxy-1,1,1,2,3,4,4,5,5,6,6,6-dodecafluor-2-(trifluormethyl)-hexane, and are subsequently washed several times with PBS.

The invention also relates to therapeutic compositions, which can be produced, in particular are produced, by means of a method according to the invention, optionally containing at least one additive, in particular marine collagen, sorbitol, mannitol, platelet-rich plasma (PRP), polyphenols, S-allyl-cysteine, pentosan-polyphosphate-Na, and/or extracellular vesicles, containing curcuminoids.

In connection with the present invention, the term “hydrogel” is understood to be a gel of water-insoluble polymer components, which can bind water and wherein the molecules, which build the gel, are chemically linked by means of covalent bonds to form a network. By means of the hydrophilic polymer components, which are present in the network, the hydrogel swells in water under significant volume increase, but without losing its material cohesion and integrity.

In connection with the present invention, the term “hydrogel particles” is understood to be a physical manifestation of the material “hydrogel”, in particular a hydrogel in particulate form.

In connection with the present invention, the term “network” is understood to be a polymer network, in particular polymer chains, which are three-dimensionally linked to one another. The polymer chains are linked to one another via crosslinking points and are preferably embodied as permanent network, wherein the polymer chains are connected to one another via chemical crosslinking points in the form of covalent bonds.

In connection with the present invention, the “concentration of sulfate or sulfonate groups of the hydrogel particles” is understood to be the number of free sulfate or sulfonate groups, expressed in mole per volume, which is present in the volume formed by the totality of the hydrogel particles. This concentration is preferably specified with the unit mmol/l. In a preferred embodiment, the concentration of sulfate or sulfonate groups is calculated by multiplying the concentration of the polyionic polymer component in the hydrogel particle in the swollen state with the number of repeating units and the number of sulfate or sulfonate groups for each repeating unit.

In connection with the present invention, a “selectively N-desulfated glycosaminoglycan” or “selectively N-desulfated heparin” is understood such that the sulfate groups bonded to nitrogen atoms of the glycosaminoglycan were removed completely or predominantly and a glycosaminoglycan having a lower sulfate group portion compared to the non-desulfated glycosaminoglycan is obtained, the lower sulfate group portion of which is based on an elimination or a reduction of the sulfate group portion of nitrogen atoms of the glycosaminoglycan.

Provided that the term “P1” is used in connection with the present application, this is preferably understood to be the concentration of sulfate or sulfonate groups, in particular the charge properties, which preferably result from the concentration of the sulfate or sulfonate groups, or in particular the global charge density, which preferably results from the concentration of the sulfate or sulfonate groups, in particular in the unit mmol/l.

The concentration of the polyionic polymer component in the hydrogel particle in the swollen state is calculated in that the concentration of the polyionic polymer, which is used during the crosslinking, is divided by the degree of swelling.

In connection with the present invention, “star-PEG” or “starPEG” is understood to be a polyethylene glycol molecule, which comprises a center with several, for example four or eight, in particular four, in particular equally long, chains, which are covalently bonded thereto.

In connection with the present invention, a “biofluid” is understood to be a fluid, which is present in a living biological system or originates therefrom. A biofluid can be, for example, a bodily fluid of a human or animal.

In connection with the present invention, a “lipopolysaccharide” is understood to be a compound of fat-like (lipo) and sugar components (polysaccharides), which can be obtained from the external membrane of gram-negative bacteria and which can act as allergen, endotoxin and/or, in particular strong, inflammatory agent.

In connection with the present invention, a “non-polymer crosslinker component” is understood to be an enzymatically cleavable peptide or short molecule, which have at least two groups, which are able to crosslink, wherein the short molecule is a molecule, which itself is not suitable for the polymerization or a monomer or an oligomer with 2 to 10 repeating units, in particular 3 to 4 repeating units.

In connection with the present invention, the term “hydrogel particles in the swollen state” or “hydrogel particles, which are present in the swollen state” are understood to be hydrogel particles, which are present in preferably physiological saline solution (PBS) and which have absorbed the maximally absorbable amount of fluid in this solution, are thus present in swollen form. In the swollen state, the hydrogel particles have thus reached their maximum volume expansion, at which they have absorbed the maximally absorbable amount of fluid. This is therefore understood to be a state of the particles, in which the particles in a pharmaceutically acceptable excipient, in particular PBS, have increased their volume compared to the volume, which is present immediately after the production. The volume swelling is preferably calculated from the volume change of the volume of the hydrogel, which is present immediately after formation of the network, compared to the volume of the swollen hydrogel, which is obtained after introduction into PBS and the swelling process, which takes place therein. The swollen state is characterized by reaching the balanced degree of swelling of the hydrogel particles in the solution, that is, preferably PBS, whereby PBS has the same ionic strength as the biofluid in the cartilage, in particular tissue fluid, in particular synovial fluid, in particular a model synovial fluid. Thermodynamically, the expansive forces (osmotic/electrostatic forces and forces based on the excluded volume of the polymer chains) and the elastic restoring forces (due to the covalently connected network chains) are balanced in this state (see also Freudenberg et al., DOI: 10.1002/adfm.201101868). In the same solution, the hydrogel particles therefore no longer change the state with respect to the degree of swelling, when they have reached the swollen state. The swollen state can preferably be established in that the hydrogel particles are incubated in a solution, in particular PBS, until their degree of swelling no longer changes, preferably for 1 h, in particular 4 h, in particular 12 h, in particular 24 h.

According to the invention, the swelling is preferably measured in that 67 μl of unpolymerized hydrogel mixture with the same chemical composition as the hydrogel particles to be produced were polymerized between two 9 mm glass slides (Menzel-Gläser, Germany), which are treated with Sigmacote (Sigma-Aldrich, Germany) for 16 h at room temperature and the resulting hydrogel discs were subsequently removed from the glass slides. The diameter of the hydrogel discs after the polymerization was optically determined by means of a scanner of the type FLA-3100 (Fujitsu, Japan) (diameter of the hydrogel immediately after the crosslinking/hydrogel formation). The hydrogel discs were subsequently swollen in phosphate-buffered saline solution (PBS), 0.9% NaCl buffered to pH 7.4 (Sigma-Aldrich, Germany), for 24 h (physiological conditions) and were determined again by means of the scanner of the type FLA-3100 (Fujitsu, Japan) (diameter in the swollen state). The swelling of the hydrogels (degree of swelling) was determined from the specific diameters of the following equation: degree of swelling=(diameter of the swollen hydrogel) 3/(diameter of the hydrogel immediately after the crosslinking/hydrogel formation) 3.

Alternatively, the degree of swelling, that is, in particular the average diameter of the hydrogel particles in the swollen state, compared to the size of the hydrogel particles immediately after the hydrogel formation, can be determined by means of fluorescence microscopy. For this purpose, the hydrogel particles are labeled immediately after the hydrogel formation and after the swelling with a fluorescent dye, preferably Atto-488-NH₂ (1% based on the number of amino groups in the hydrogel). The particle sizes are captured and measured via image evaluation software by means of a fluorescence microscope.

With the same composition of the hydrogel discs and hydrogel particles, the degree of swelling determined by means of hydrogel discs (x-times the size between swollen state and state of the hydrogel particles immediately after the hydrogel formation) corresponds to the degree of swelling of the hydrogel particles, which was determined by means of fluorescence microscopy.

In connection with the present invention, the “average mesh width” is understood to be the average distance between two network node points (see Polymer Physics, Michael Rubinstein and Ralph H. Colby, 2006, Oxford University Press, Oxford). In certain approximation, the distance corresponds to the sterical resistance of the network for transport processes of molecules through the network because molecules, which have a larger dimension than the mesh width, are eliminated from a penetration of the network for steric reasons. The mesh width considers an ideal network without any defect structures. The storage modulus of the hydrogel particles in the swollen state, which is determined experimentally, in particular by means of oscillatory rheometry, can derive the average mesh width of the hydrogels on the basis of the rubber elasticity theory and the assumptions of an ideal network without defects with the help of the following formula (Polymer Physics, Michael Rubinstein and Ralph H. Colby, 2006, Oxford University Press, Oxford):

$$ξ = {( \frac{{G}^{′} {N}_{A}}{R T} )}^{- 1 / 3}$$

whereby G′ is the measured storage modulus in Pa, N_{A} is the Avogadro constant (6.023×10⁻²³ mol⁻¹), R is the universal gas constant (8.314472 m³ Pa K⁻¹ mol⁻¹) and T is the absolute temperature (in Kelvin; measurement is preferably performed at 295 K).

In connection with the present invention, the “friction coefficient” is understood to be a measurement variable, which specifies the ratio of frictional force to contact force. This measurement variable can be measured in particular in a tribometry cell, in particular an Anton Paar MCR 301rheometer, in particular in an oscillating rheometer at a speed of 10 revolutions per second with a soda-lime glass ball (12.7 mm diameter), which is pressed onto a silicon layer with a normal force of 10 N.

The fluid/composition to be examined (in particular 1 ml) is thereby placed into the tribometry cell and forms a lubricating film between glass ball and silicon layer and thus reduces the sliding friction compared to a measurement with pure water. The friction coefficient is determined in the method by means of the device software with a normal force of 10 N and a rotational speed of 10 revolutions per second. A rheometer MCR 301 from Anton Paar with a tribometry cell of the type T-PTD 200 from the same company is preferably used.

In connection with the present invention, the term “cytokine” is understood to be proteins, which regulate the growth and/or the differentiation of cells. Some cytokines are growth factors, others play an important role for immunological reactions and during inflammation processes and are also referred to as mediators. According to the invention, cytokines are preferably understood to in particular be interferons, interleukins, colony-stimulating factors, tumor necrosis factors and “chemokines”, thus small signal molecules.

In connection with the present invention, the “reduction of the concentration of cytokines, in particular chemokines”, is understood such that the concentration of free cytokines, in particular chemokines, is reduced in a synovial fluid, in particular a model synovial fluid. Without being bound to the theory, this takes place essentially by bonding the cytokine, in particular chemokine, to the hydrogel particles of the compositions according to the invention, in particular by means of sequestering.

The reduction is preferably measured in that a predetermined quantity of cytokine, in particular chemokine, is placed into a synovial fluid, in particular a model synovial fluid, of a predetermined volume and wherein a material to be analyzed, in particular the present therapeutic composition, in particular the hydrogel particles, are present in this synovial fluid, in particular the model synovial fluid.

To measure the reduction, 50 μl of the therapeutic composition to be analyzed are preferably incubated in 0.5 ml reaction vessels, in particular Protein LoBind® reaction vessels (Eppendorf Tubes, Germany), the composition is located on the bottom of the reaction vessel—with 250 μl of a protein or protein mixture solution for 24 h at room temperature. The protein or protein mixture solution is produced by dissolving one or several proteins (cytokines, chemokines or other signal molecules) in PBS with 1% of bovine albumin and 0.05% (m/v) of ProClin™ 300 (Sigma-Aldrich), in order to attain a respective active concentration of 10 ng/ml of the proteins. As reference, the same protein or protein mixture solution without presence of the 50 μl of the therapeutic composition is likewise incubated in 0.5 ml reaction vessels, in particular Protein LoBind® reaction vessels, for 24 h at room temperature. A respective sample of the solution (supernatant without composition) is subsequently removed from the reaction vessels, in particular Protein LoBind® reaction vessels, and the protein concentrations are measured by means of ProcartaPlex™ (Thermo Fisher, Germany) in combination with the corresponding ProcarteaPlex™ Simplex Kits on a device of the type Bioplex 200 (Biorad, Germany).

The reduction of the concentration of cytokines, in particular chemokines, is determined from the determined concentrations of the solution, which was incubated with the therapeutic composition to be analyzed and without this composition, according to the following equation: reduction in %=(1−concentration in solution with composition/concentration in solution without composition)×100.

In connection with the present invention, a “model synovial fluid” is understood to be a fluid, which has at least one cytokine, in particular chemokine, in a concentration of 10 ng/ml, dissolved in a solution of 1 mg/ml of bovine albumin in PBS.

In connection with the present invention, the “injection force” is understood to be the force, which is necessary to push a composition through a 25-gauge needle, thus a needle with an outer diameter according to EN ISO 9626 of 0.5 mm, at a speed of 0.05 ml/s. In preferred embodiment, the injection force is determined according to the procedure and measurement rule according to the example C).

The injection force is preferably determined in that 0.5 ml of the composition is filled into a 1 ml syringe and the necessary force for extrusion through a 25 G syringe needle is measured in a ZwickRoell universal testing machine with a 50 N measuring cell at 10 ml/s.

In connection with the present invention, the “storage modulus” of the hydrogel particles is understood to be the elastic portion of the complex shear modulus. The elastic portion is proportional to the portion of the deformation energy, which is stored in the material and which can be obtained from the material again after release. This energy is preferably determined on its chemically/physically identical hydrogel discs by means of oscillatory rheometry in a plate/plate arrangement by means of frequency-dependent measurement of the shear modulus. The storage moduli specified according to the invention are the storage moduli of hydrogel particles swollen in physiological saline solution (PBS). In preferred embodiment, the storage modulus is determined according to the procedure and measurement specification according to the example B).

In connection with the present invention, the term “crosslinker” is understood to be the formation of covalent connections between polyionic polymer component with at least one uncharged polymer component or a non-polymer crosslinker component, whereby the components are preferably mixed with one another for this purpose.

In connection with the present invention, the term “arthritis” is understood to be a cartilage disease or joint disease, in particular a cartilage joint disease or joint cartilage disease. The arthritis is in particular an inflammatory disease of a joint and/or cartilage. The inflammation can be traced back to an infection, for example a bacterial, viral or fungal infection, an immune reaction, in particular an autoimmune reaction, a metabolic disorder, for example gout, or a mechanical cause, in particular a wearing of joint structures, in particular cartilage tissue, or traumatic influences, for example an injury or an accident, on joint structures, in particular cartilage tissue. The inflammation can have an acute or chronic progression.

Arthritis tracing back to mechanical influences, thus, for example, to accidents or/and chronic-degenerative processes, will also be referred to as osteoarthritis here (arthrosis or activated arthrosis).

In connection with the present invention, the term “arthritis” is in particular understood to be osteoarthritis (arthrosis or activated arthrosis), rheumatoid arthritis, infectious arthritis (septic arthritis), post-infectious arthritis, psoriatic arthritis and gouty arthritis.

In connection with the present invention, the term “arthritis” is in particular understood to be osteoarthritis (OA) and rheumatoid arthritis (RA). Osteoarthritis (also referred to as arthrosis or as activated arthrosis, respectively), is in particular a degenerative, in particular chronic-degenerative, joint change with cartilage degradation, which traces back to destructive mechanical influences on the cartilage, leads to inflammations and is often associated with pain, swelling and functional limitations. The inflammatory joint disease rheumatoid arthritis (chronic polyarthritis, primary chronic polyarthritis, RA) is a chronic autoimmune disease and is associated with identical or similar clinical symptomatology. It also includes Morbus Bechterew (Spondylitis ankylosans).

The arthritis can appear in all areas of the human or animal body, in which joints and cartilage are present, in particular the toes, fingers, knee, hip, spine, in particular neck or back spine, shoulder, elbows and wrists.

According to the invention, the arthritis is associated with the symptoms of pain, swelling, functional limitations and restrictions of movement of the affected joints, damages to cartilage structures, increase of the concentrations of inflammatory cytokines and inflammation markers and/or pathological immune responses.

In one embodiment, an arthritis of the back spine can appear with the symptoms of lower back pain.

In particularly preferred embodiment, a therapy of the arthritis according to the present invention leads to a reduction or elimination at least of a portion of the clinical symptomatology of an arthritis, in particular a decline or prevention of swelling in the joint area, a relief or prevention of pain, a reduction or prevention of restrictions of movement as well as functional limitations, a reduction or prevention of inflammation processes, in particular a reduction of the concentration of cytokines, which have an inflammatory effect, and/or pathological immune reactions.

In a preferred embodiment, an advantageous effect of the present invention is present when a swelling of the knee joint is prevented in the mouse model after injection of 100 ng of lipopolysaccharides (LPS), measured by the swelling of the knee by means of caliper 72 h after the LPS injection.

In connection with the present invention, the term “at least one” is understood to be a quantity, which expresses a number of 1 or 2 or 3 or 4 or 5 or 6 or 7 or 8 or 9 or 10 and so on. In a particularly preferred embodiment, the term “at least one” can represent exactly the number 1. In a further preferred embodiment, the concept “at least one” can also mean 2 or 3 or 4 or 5 or 6 or 7.

Provided that quantitative information, in particular percentages, of components of a product or of a composition is specified in connection with the present invention, it adds up to 100% of the composition and/or of the product, unless explicitly specified otherwise or unless an expert analysis shows otherwise, together with the other further components of the composition or of the product, which are explicitly specified or shown by expert analysis.

Provided that a “presence”, a “containing”, a “having” or a “content” of a component is mentioned expressly or is implied, this means that the respective component is present, in particular in measurable quantity.

Provided that a “presence”, a “containing” or a “having” of a component in a quantity of 0 [unit], in particular mg/kg, μg/kg or % by weight, is mentioned explicitly or is implied in connection with the present invention, this means that the respective components are not present in a measurable quantity, in particular are not present.

The number of the specified decimal places corresponds to the precision of the respective applied measuring method.

In connection with the present invention, the term “and/or” is understood such that all members of a group, which are connected by the term “and/or” are disclosed alternatively to one another as well as in each case cumulatively among one another in any combination. For the expression “A, B and/or C”, this means that the following disclosure content is to be understood by it: a) A or B or C or b) (A and B), or c) (A and C), or d) (B and C), or e) (A and B and C).

In connection with the present invention, the terms “comprising” and “having” is understood such that in addition to the elements, which are captured explicitly by these terms, further elements, which are not mentioned explicitly, can be included. In connection with the present invention, these terms are also understood such that the explicitly mentioned elements alone are captured and that no further elements are present. In this special embodiment, the meaning of the terms “comprising” and “having” is synonymous with the term “consisting of”. The terms “comprising” and “having” furthermore also capture compositions, which, in addition to the explicitly mentioned elements, also include further non-mentioned elements, which, however, are of a functional and qualitatively subordinate nature. In this embodiment, the terms “comprising” and “having” are synonymous with the term “essentially consisting of”. The term “consisting of” means that the explicitly mentioned elements alone are present and that the presence of further elements is ruled out.

Further embodiments of the present invention are the subject matters of the subclaims and of further dependent claims.

The invention will be described in more detail on the basis of the following example and the corresponding figures.

The figures show:

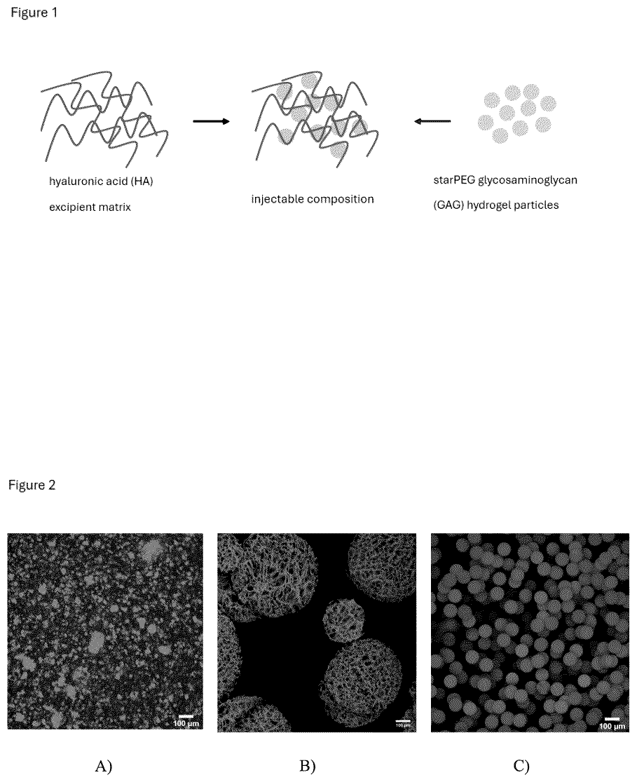

FIG. 1 schematically shows the structure of a therapeutic composition according to the invention,

FIG. 2 shows photographic illustrations of different hydrogel particles used according to the invention,

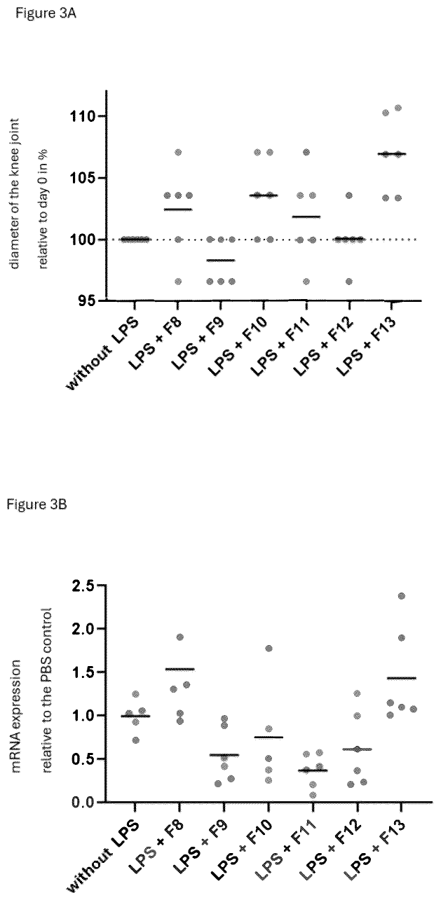

FIG. 3A shows a graphic illustration of the reduction of the LPS-induced swelling of the knee joint in the mouse model and

FIG. 3B shows a graphic illustration of the gene expression of the inflammation marker TNFa.

The sequence listing shows:
SEQ ID No. 1 matrix metalloproteases (MMPs)-responsive sequence PQGIWGQ,
SEQ ID No. 2 matrix metalloproteases (MMPs)-responsive sequence IPVSLRSG,
SEQ ID No. 3 matrix metalloproteases (MMPs)-responsive sequence VPMSMRGG,
SEQ ID No. 4 elastase-responsive sequence AAPV,
SEQ ID No. 5 elastase-responsive sequence APEEIMDRQ,
SEQ ID No. 6 thrombin-responsive sequence GGF-pipecolic acid-RYSWGCG,
SEQ ID No. 7 thrombin-responsive sequence GG-cyclohexylalanine-ARSWGCG,
SEQ ID No. 8 FXa-responsive sequence GGIEGRMGGWCG,
SEQ ID No. 9 kallikrein-responsive sequence CGGGPFRIGGWCG,
SEQ ID No. 10 aureolysin-responsive sequence ADVFEA,
SEQ ID No. 11 aureolysin-responsive sequence AAEAA,
SEQ ID No. 12 protease IV-responsive sequence MKATKLVLGAVILGSTLLAG,
SEQ ID No. 13 matrix metalloproteases (MMPs)-responsive sequence GPQGIAGQ,
SEQ ID No. 14 matrix metalloproteases (MMPs)-responsive sequence GPQGIWGQ,
SEQ ID No. 15 matrix metalloproteases (MMPs)-responsive sequence GCGGPQGIWGQGGCG.

### EXAMPLE

A therapeutic composition of the present invention according to the invention, which is suitable for the intra-articular injection, has, in addition to a hyaluronic acid component, hydrogel particles and at least one pharmaceutically acceptable excipient, the production and use of which will be described below. The basic structure of this therapeutic composition is illustrated in FIG. 1, which shows the two therapeutically effective elements, namely the hyaluronic acid component and the star-PEG/glycosaminoglycan (heparin) network forming the hydrogel particles.

### A) Hyaluronic Acid Component

A hyaluronic acid (sodium salt) from the company Contipro, molecular weight >1.9 Mio. Da was used. The purity corresponds to pharmaceutical quality and a certification in accordance with ISO 13485 (medical product). The concentrations in the composition are named in paragraph C).

A particularly advantageous range of the molar mass of the hyaluronic acid lies in the range from 500 kDa to 100 Mio Da.

### B) Production of the Hydrogel Particles

### B1: Description of the Hydrogel Properties

The hydrogel according to the invention consists of charged building blocks, thus the polyionic polymer component, such as glycosaminoglycan, in particular heparin (building block A1) or selectively N-desulfated heparin (building block A2) and uncharged building blocks, thus non-polymer crosslinker component and/or the uncharged polymer component, for example uncharged polymers in the form of multi-arm polyethylene glycols (PEG) in amine-terminated (building block B) and/or carboxy-terminated (building block C) form, see Table 2. The charged and uncharged building blocks are thereby covalently networked into a polymer network, which can preferably be obtained by the activation of the carboxyl groups of the charged building block with 1-ethyl-3-(3-dimethylaminopropyl) carbodiimide (EDC)/N-hydroxysulfosuccinimide (S-NHS) and either a direct crosslinking with the uncharged building block containing amino groups or via crosslinker molecules (linkers) with at least two amino groups, each by forming amide. The synthesis of selectively N-desulfated heparin takes place according to the synthesis rule from Atallah et al. (Doi: 10.1016/j.biomaterials.2018.07.056). For the variation of the charge properties (in particular of the parameter P1, the global charge density), a second uncharged building block with carboxyl groups, (for example building block C, 8-ArmPEG, carboxy-terminated), which, just like the charged building block, is activated via EDC/S-NHS at the carboxyl groups, can furthermore be used as third building block of the hydrogel network. The network is then formed via crosslinking between building block A and building block C on the one hand and building block B (amine-terminated) on the other hand.

The structures of hydrogels preferred according to the invention are defined by the molar concentrations of the building blocks A to C in the hydrogel mixture during the crosslinking (see Table 1). These concentrations describe the concentration of the network-forming building blocks immediately after the mixing in the reaction state. From these concentrations, the molar ratios of the hydrogel building blocks during the crosslinking can be calculated (see Table 1). The network formation takes place over a time period of 12 hours, whereby the hydrogel formed after this time period corresponds to the hydrogel state after the crosslinking, in particular after method step b).

To produce the hydrogels of a type 1-9, the components A-C are for this purpose dissolved for 5 min by treatment in ultrasound (the initial concentrations are thereby selected, so that after mixing the 1 to 3 parts by volume (for component A to C) in the final reaction mixture, the concentration of the building blocks A to C results according to the information in Table 1). The activation reagents EDC/sNHS are dissolved stoichiometrically in ultrapure water in the ratio of the amino groups, which are present in the reaction mixture (4-times molar concentration of the building block B due to the molecular architecture with the 4-arm PEG) as follows, so that 2 mol of EDC per mol of amino group and 1 mol of sNHS proportional to 1 mol of amino groups result in the final reaction mixture. The activation reagents are then combined with the components A and C, which carry the carboxyl groups, and are activated for 10 min at room temperature. The addition of the component B to the mixture of A+C+activation reagents takes place subsequently in order to initiate the covalent network formation of the material.

For the following calculations, it is assumed that all network building blocks are incorporated quantitatively into the hydrogel and remain in the hydrogel during the subsequent balanced swelling step in PBS. The volume swelling is calculated from the volume change of the test bodies after network formation and the subsequent swelling in PBS.

The swelling was measured in that 67 μl of unpolymerized hydrogel mixture with the same chemical composition as the hydrogel particles to be produced are polymerized between two 9 mm glass slides (Menzel-Gläser, Germany), which were treated with Sigmacote (Sigma-Aldrich, Germany) for 16 h at room temperature and the resulting hydrogel discs were subsequently removed from the glass slides. The diameter of the hydrogel discs after the polymerization was optically determined by means of a scanner of the type FLA-3100 (Fujitsu, Japan) (diameter of the hydrogel immediately after the crosslinking/hydrogel formation). The hydrogel discs were subsequently swollen in phosphate-buffered saline solution (PBS), 0.9% NaCl buffered to pH 7.4 (Sigma-Aldrich, Germany), for 24 h (physiological conditions) and were determined again by means of the scanner of the type FLA-3100 (Fujitsu, Japan) (diameter in the swollen state). The swelling of the hydrogels was determined from the specific diameters of the following equation: swelling=(diameter of the swollen hydrogel) 3/(diameter of the hydrogel immediately after the crosslinking/hydrogel formation) 3.

Only the hydrogels swollen in PBS are used for the further characterization (rheometry for determining the storage modulus, sequestering of IL-8 and further inflammatory proteins) and the preparation of mixtures. For this reason, the charge properties of the hydrogels (the sulfate/sulfonate concentration P1) can be calculated from the reaction mixtures by using the concentration of the charged building blocks in the reaction mixture and the volume swelling degree (see Table 1, column H). The calculation was made from the molar concentration of the hydrogel building blocks during the hydrogel formation (see Table 1, column A) in consideration of the volume swelling by assuming the complete installation of the polymer hydrogel building blocks.

For this purpose, the concentration of the polyionic polymer component in the swollen hydrogel (Table 1, column G) was first calculated from the concentration of the polyionic polymer, which is used for the hydrogel formation (Table 1 column A), divided by the degree of swelling (Table 1, column E). The concentration of the sulfate or sulfonate groups in the swollen hydrogel (parameter P1, column H, Table 1) was calculated from the concentration of the polyionic polymer component (the charged building block) in the swollen hydrogel (Table 1, column G), multiplied by the number of the sulfate/sulfonate groups per polymer molecule (Table 2).

The hydrogel materials are characterized by the mixing ratios according to Table 1 (whereby the formation of an elastic hydrogel serves as the most significant criterion, that is, an elastic hydrogel results after the swelling in PBS, which hydrogel does not dissolve and is preferably characterized by a range of 0.1 to 22 kPa for the storage modulus, which correlates directly with the elasticity/stiffness of the network (determined by rheometry). The storage modulus of the hydrogel particles was determined by means of oscillatory rheometry (in kilo pascal) by means of a shear rheometer of the type Ares from the company TA Instruments United Kingdom. For this purpose, completely swollen hydrogel discs (swollen for 24 h in PBS) punched out to a diameter of 8 mm were measured in a 9 mm plate/plate measuring arrangement with increasing frequency of 1-100 rad/s at room temperature with small deformation (2%) and the average value was determined over the entire frequency range (1 measuring value on one sample). The reported values are the average values of four hydrogel discs, which are produced independently of one another, and are specified +/−the standard deviation (SD).

In addition to the storage modulus, the charge density in the hydrogel, expressed via the concentration of the anionically charged sulfate/sulfonate groups in the swollen hydrogel (see parameter P1 in Table 1, column H, calculation see above), serves as second important parameter.

For this purpose, a parameter variation of P1 of 0.1 mmol/l to 800 mmol/l is provided, particularly advantageous ranges are 20 to 500 mmol/l, in particular 50 to 200 mmol/l.

TABLE 1
Formation rule and chemical-physical description of the hydrogels, types 1 to 12

| | | | | | | Hydrogel network after balanced swelling | | | | | |
|---|---|---|---|---|---|---|---|---|---|---|---|
| | Hydrogel mixture during the | | | | | in physiological saline solution (PBS) | | | | | |
| | network formation, non-swollen | | | | | | | Concentration | | | |
| | Concentration | | Concentration | | | | | of the | Concentration | | |
| | of polyionic | Type of the | of uncharged | | | | | polyionic | of sulfate/ | Storage | |
| | polymer | polyionic | polymer | | Ratio | Swelling in % | | polymer | sulfonate | modulus in kPa | |
| | component | polymer | component | | of the | Average | | component | groups (P1) | Average | |
| | in mmol/l | component | in mmol/l | | components | value | SD | in mmol/l | in mmol/l | value | SD |
| Name | A | | B | C | D | E | F | G | H | I | J |
| Type 1 | 5.56 | A1 | 5.56 | 0.00 | 1.0 | 5.16 | 0.04 | 1.08 | 75.4 | 2.97 | 0.36 |
| Type 2 | 3.92 | A1 | 7.84 | 0.00 | 2.0 | 2.66 | 0.05 | 1.47 | 103.1 | 3.60 | 0.20 |
| Type 3 | 3.03 | A1 | 9.09 | 0.00 | 3.0 | 2.23 | 0.04 | 1.36 | 95.0 | 4.95 | 0.24 |
| Type 4 | 1.50 | A1 | 5.56 | 4.06 | 1.0 | 2.01 | 0.02 | 0.75 | 52.2 | 0.46 | 0.01 |
| Type 5 | 1.50 | A1 | 7.84 | 2.42 | 2.0 | 1.55 | 0.02 | 0.97 | 67.6 | 1.30 | 0.09 |
| Type 6 | 1.50 | A1 | 9.09 | 1.53 | 3.0 | 1.47 | 0.01 | 1.02 | 71.4 | 6.60 | 0.17 |
| Type 7 | 0.50 | A1 | 7.84 | 5.06 | 1.0 | 2.06 | 0.05 | 0.24 | 17.0 | 1.95 | 0.10 |
| Type 8 | 0.50 | A1 | 7.84 | 3.42 | 2.0 | 1.73 | 0.04 | 0.29 | 20.2 | 3.06 | 0.04 |
| Type 9 | 0.50 | A1 | 9.09 | 2.53 | 3.0 | 1.72 | 0.11 | 0.29 | 20.4 | 9.88 | 0.34 |
| Type 10 | 5.00 | A2 | 5.00 | 0.00 | 1.0 | 1.89 | 0.06 | 2.64 | 124.33 | 1.829 | 0.392 |
| Type 11 | 2.50 | A2 | 5.00 | 2.50 | 2.0 | 3.09 | 0.18 | 1.61 | 76.05 | 0.423 | 0.052 |
| Type 12 | 1.50 | A2 | 5.00 | 3.50 | 3.3 | 3.88 | 0.15 | 1.28 | 60.56 | 0.645 | 0.063 |

TABLE 2
Chemical-physical properties of the polymer hydrogel
building blocks (components); MWP: molar mass of the
polymer, MWWE: molar mass of the repeating unit (WE)

| | | | | | Number of |
|---|---|---|---|---|---|
| | Hydrogel | | | Number of | sulfates |
| | building | MWP in | MWWE in | sulfates | per polymer |
| Polymer | block | g/mol | g/mol | per WE | molecule |
| Heparin | A1 | 14,000 | 540 | 2.7 | 70 |
| Selectively | A2 | 13,600 | 523 | 1.8 | 47 |
| N-desulfated | | | | | |
| heparin | | | | | |
| 4-arm PEG, | B | 10,000 | 44 | 0 | 0 |
| amine- | | | | | |
| terminated | | | | | |
| 8-arm PEG, | C | 10,000 | 44 | 0 | 0 |
| carboxyl- | | | | | |
| terminated | | | | | |

### B 2) Production of Hydrogel Particles

The hydrogel networks corresponding to the formation rule according to B1) can preferably be processed into hydrogel particles in the preferred size range by means of three different methods. Target parameters are thereby the average particle size (average diameter) prevailing in the final particle suspension with a diameter of maximally 200 μm and the percent by volume of the particles, whereby a concentration of the particle suspension by means of centrifugation is advantageous.

The production of the particles can be achieved by means of method B2.1) from volume gel materials produced according to the method according to B1) by means of suitable mechanical comminution, for example grinding, shredding, high pressure or ultrasonic treatment of the hydrogels. The ultrasonic comminution is advantageously used for this purpose, during which hydrogels swollen completely in PBS according to method B1) are treated as gel bodies in a fivefold volume of PBS for 10 min at full power in a Bandelin-Sonoplus ultrasonic homogenizer (Germany). The particle mixture can subsequently be filtered through a filter with an average pore size of 200 μm, in order to separate larger fragments, which are still present. Other pore sizes of filters are possible. FIG. 2A shows hydrogel particles of this type, which are obtained by means of mechanical comminution from hydrogels.

The particle size (average diameter) of maximally 200 μm is significant in order to ensure an injectability of the composition (see C), a particularly advantageous range is realized by the range of 80 to 10 μm.

The production of the particles can alternatively take place by means of method B2.2 by means of suitable microfluidic methods, in particular co-flow methods, as follows: for this purpose, the solutions of the components A+C+EDC/sNHS and of the component B (concentrations and mixture according to the formation rule of B1) are pre-mixed at 4° C. and are subsequently dispersed into small drops at a flow speed of 10-20 μl/min by means of a microfluidic chip, which specifies the size criteria (diameter) with crossover arrangement (an aqueous phase input for the cooled gel mixture and an input for the organic phase) at a flow speed of 10-20 μl/min (the aqueous gel phase) into an organic phase of 3M™ Novec™ 7500 Engineered Fluid (3-ethoxy-1,1,1,2,3,4,4,5,5,6,6,6-dodecafluor-2-(trifluoromethyl)-hexane) and 1% of PFPE-PEG-PFPE-surfactant (Krytox™ 157 FSH (DuPont)-Jeffamine™ ED 600 Amine (Huntsman)-Krytox™ 157 FSH (DuPont), which can be obtained according to the synthesis rule published in Lab Chip, 2008, 8, 1632-1639). The geometry of the microfluidic chip as well as the ratios of the flow speed of the two phases determine the final particle size. The formed drops react within the flow path and by means of subsequent dwell time in a collection container for a time period of 1-12 hours and are subsequently separated by adding a percent by volume of 1:1 between 1H, 1H,2H,2H-perfluoro-1-octanol and Novec™ 7500 phase and are subsequently cleaned and swollen completely by means of repeated washing in PBS.

Spherical particles with narrow size distribution in the range of deviations <10% of the average particle diameter form by means of this method. A filtration or further post-processing is not necessary. FIG. 2C shows hydrogel particles of this type obtained by means of a microfluidic device.

The particle size (average diameter) of maximally 200 μm is significant in order to ensure an injectability of the composition (see C)), the range of 80 to 10 μm forms a particularly advantageous range.

FIG. 2B shows hydrogel particles obtained by means of cryogelation and subsequent lyophilization (method B2.3).

In all method variations, in particular in the case of both methods B2.1 and B2.2, the percent by volume of the hydrogel particles can be concentrated in the suspension by means of centrifugation of the particle suspension for 5 min at 3000G and the supernatant can be removed. The concentrated particle suspension is subsequently absorbed by means of suitable pipettes (for example positive displacement pipettes) and is used to produce the mixtures according to paragraph C).

### C) Production of the Therapeutic Composition of Hyaluronic Acid and Sequestering Hydrogel Particles

The therapeutic composition for use in the case of arthritis, in particular osteoarthritis, consists of a mixture of hydrogel particles, pharmaceutically acceptable excipient and hyaluronic acids. FIG. 1 schematically shows an injectable therapeutic composition of the preset invention produced from a hyaluronic acid excipient matrix and star-PEG glycosaminoglycan (GAG) hydrogel particles.

The composition (referred to as F1-F7, see Table 3) is produced by mixing variable percentages by volume of the three components 1) hyaluronic acid, 2) hydrogel particle suspension and 3) PBS (see Table 3). A solution with 5 (g/g) % of hyaluronic acid in PBS is used thereby for the component hyaluronic acid (produced by means of dissolution and homogenization by stirring the powdery hyaluronic acid in PBS). The component 2 (hydrogel particle suspension) is specified according to the hydrogel formation rules according to B1 (types 1-8, see Table 1) and a production process according to B2 (preferably B2.1). The component 3) PBS can optionally have sorbitol or other antioxidative substances. The mixing of the components 1) to 3) takes place according to the percentages by volume, which are specified in Table 3, by means of suitable homogenization (for example by stirring for 10 min at a stirring speed of 500 U/min).

Particularly advantageous mechanical properties of the composition are an easy injectability (characterized by a low injection force) and simultaneously an advantageous high lubrication effect (characterized by a low friction coefficient).

To determine the injectability, the injection force was measured as follows: 0.5 ml of the therapeutic composition was filled into a 1 ml syringe and the necessary force for extrusion through a 25 G syringe needle was measured in a ZwickRoell universal testing machine with a 50 N measuring cell at 10 ml/s. The lubrication effect was measured in a tribometry test setup with a soda-lime glass ball (12.7 mm diameter) on a silicon layer in an Anton Paar MCR 301 rheometer with a T-PTD 200 measuring cell at a speed of 10 revolutions per second and an applied normal force of 10 N.

In an exemplary manner, an injection force of 3.1±0.4 N, which is slightly smaller than the injection force for the 1% hyaluronic acid alone (the excipient matrix) with 3.7±0.8 N and which is not too removed from the injection force of pure water with 2.4±0.4 N, were measured for the compositions F2, F4 and F6. A friction coefficient in the range of 0.091±0.004, which is comparable with the friction coefficient for the 1% hyaluronic acid alone with 0.154±0.002 and significantly smaller than the friction coefficient of water with 0.644±0.157, were likewise determined for the friction coefficients for the compositions F2, F4 and F6.

An injection force of <10 N (test procedure see above) and a lubrication effect, which can be described by a friction coefficient <0.2 was achieved.

As significant property, the composition additionally has the potential for sequestering at least one of the chemokines IL-8, IP-10, MCP-1, MIP-1a, MIP-1ß and/or RANTES, which act in a pro-inflammatory manner. The composition is characterized in that it bonds at least one (or all) of the factors from the group of the chemokines, which act in a pro-inflammatory manner, to a certain percentage of an application-relevant solution.

To characterize the bond of these chemokines, an artificial synovial fluid is produced by mixing recombinant chemokines in the concentration range of approx. 10 ng/ml of the respective chemokines in a 0.1% albumin solution (BSA) in PBS. The composition (in each case 0.5 ml) is then brought into contact with a volume of 0.5 ml of artificial synovial fluid for 24 hours at room temperature and the supernatant is subsequently removed and the depletion of chemokines in the supernatant compared to an untreated control solution is determined by means of multiplex immunoassay. The results are illustrated in Table 3 and show a graduated depletion as a function of the gel type (type 2, type 5 or type 8) and of the formulation (F1-F7).

After exact determination of the parameters P1 for all gel types, a further correlation of the sequestering was performed with the hydrogel composition and the composition of the mixture. The formulation F3 has a particularly advantageous strong sequestering of the chemokines IL-8, IP-10, MCP-1, MIP-1a, MIP-1ß and RANTES with >64% at a simultaneously advantageous low injection force of 0.03 N and an advantageous low friction coefficient of 0.053 (Tables 3 and 4).

A strong sequestering of pro-inflammatory chemokines from artificial synovial fluid was achieved by means of the composition according to the invention of significantly more than 50%. Table 5 shows essential properties of the therapeutic composition according to the invention in outline format.

To characterize the anti-inflammatory and relieving effect of the composition on an intra-articular inflammation, 100 ng of lipopolysaccharides dissolved in 10 μl of physiological saline solution (PBS) were injected into the knee joint (i.e. intra-articularly) of C57BL/6J mice in order to induce a joint inflammation. After 24 h, 4 μl of the compositions F8, F9, F10, F11, F12 or F13 (only PBS, control) were injected intra-articularly into the knee joint, which was treated with LPS injection.

Knee joints, which were only treated with physiological saline solution (PBS) (without LPS induction) served as additional control group. Prior to the LPS injection as well as 72 h after the initial LPS injection, the diameter of the knee joint was captured by means of caliper as measure for the swelling of the knee, which reflects the clinical symptomatology in the case of human joint inflammations and swellings very well. The change of the joint swelling is expressed as a percentage relative to the initial value prior to the LPS-induced inflammation and is standardized to the additional control group with pure PBS injection (FIG. 3A). The gene expression of the inflammation marker TNFa (tumor necrosis factor alpha) was likewise analyzed after 72 h by means of rt-qPCR (quantitative PCR), which indicates the presence and activation of immune cells (FIG. 3 B).

The formulation F9 has a particularly advantageous, strong anti-inflammatory and symptom-relieving effect with a knee swelling with a value of 98.3% and a gene expression of the inflammation marker TNFa of 55.0% relative to the value without induction of the inflammation (see Table 3 and FIG. 3A). In the case of this effect, the hyaluronic acid component of the composition plays a positive role and causes a surprising additive effect on the efficacy because the same composition without hyaluronic acid (F10) has a smaller anti-inflammatory and lower effect relieving the knee swelling (knee swelling at 103.6%, gene expression of TNF at 75.6%) (see Table 3 and FIG. 3 B).

TABLE 3
Chemokine bond of different therapeutic compositions

| | | Percent by volume in | | | | | |
|---|---|---|---|---|---|---|---|
| | | the final composition | | | | | |
| | | 10% by | | | Final mass | | |
| | Type | weight of | Hydrogel | | concentration of | | |
| | of the | hyaluronic | particles | | the hyaluronic | | |
| | hydrogel | acid | (according | | acid in the | Chemokine bond in vitro | |
| Composition | particles | solution | to B2.1) | PBS | composition | IL-8 | IP-10 |
| F1 | Type 2 | 10% | 10% | 80% | 1% | 40.87% | 82.83% |
| F2 | Type 2 | 10% | 20% | 70% | 1% | 68.97% | 92.13% |
| F3 | Type 2 | 10% | 50% | 40% | 1% | 82.74% | 94.24% |
| F4 | Type 5 | 10% | 20% | 70% | 1% | 52.98% | 63.75% |
| F5 | Type 8 | 10% | 10% | 80% | 1% | 23.55% | 41.94% |
| F6 | Type 8 | 10% | 20% | 70% | 1% | 35.31% | 69.31% |
| F7 | Type 8 | 10% | 50% | 40% | 1% | 44.45% | 50.31% |
| F8 | Type 10 | 10% | 20% | 70% | 1% | — | — |
| F9 | Type 10 | 10% | 50% | 40% | 1% | 66.40% | 33.51% |
| F10 | Type 10 | 0% | 50% | 50% | 0% | 71.75% | 46.22% |
| F11 | Type 11 | 10% | 50% | 40% | 1% | — | — |
| F12 | Type 12 | 10% | 50% | 40% | 1% | — | — |
| F13 | — | 0% | 0% | 100% | 0% | — | — |

| | | | | | Anti-inflammatory effect in the | |
|---|---|---|---|---|---|---|
| | | | | | knee joint in the mouse model | |
| | | | | | Relative | Gene expression |
| | | | | | joint swelling | of TBFa |
| | Chemokine bond in vitro | | | | after 72 h | after 72 h |
| | | MIP- | MIP- | | relative to the | relative to the |
| Composition | MCP-1 | 1alpha | 1beta | RANTES | PBS control | PBS control |
| F1 | 65.38% | 31.91% | 28.76% | 88.73% | — | — |
| F2 | 82.05% | 45.71% | 44.76% | 94.57% | — | — |
| F3 | 88.39% | 64.31% | 66.13% | 96.55% | — | — |
| F4 | 65.69% | 29.69% | 51.43% | 75.16% | — | — |
| F5 | 40.47% | 21.38% | 26.51% | 77.60% | — | — |
| F6 | 55.90% | 26.44% | 35.69% | 83.16% | — | — |
| F7 | 50.13% | 23.76% | 40.49% | 79.81% | — | — |
| F8 | — | — | — | — | 102.4% | 153.8% |
| F9 | 67.86% | 14.29% | 21.77% | 52.26% | 98.3% | 55.0% |
| F10 | 62.52% | 33.83% | 20.12% | 59.62% | 103.6% | 75.6% |
| F11 | — | — | — | — | 101.8% | 37.3% |
| F12 | — | — | — | — | 100.0% | 61.7% |
| F13 | — | — | — | — | 106.9% | 143.7% |

TABLE 4
injection force and friction coefficient

| | Type of the | | Friction coefficient |
|---|---|---|---|
| | hydrogel | Injection | at 10 revolutions |
| Composition | particles | force in N | per second |
| F1 | type 2 | 0.032 | 0.088 |
| F2 | type 2 | 0.028 | 0.059 |
| F3 | type 2 | 0.030 | 0.053 |
| F4 | type 5 | 0.031 | 0.032 |
| F5 | type 8 | 0.030 | 0.075 |
| F6 | type 8 | 0.032 | 0.053 |
| F7 | type 8 | 0.032 | 0.046 |

TABLE 5
overview of some properties of the therapeutic
composition according to the invention:

| | Effect | Quantification |
|---|---|---|
| 1 | Bond of inflammatory | Bond >50% of the chemokines |
| | chemokines | (according to the test |
| | | arrangement paragraph C) |
| 2 | Injectability through | Injection force <10N (measured |
| | 25 gauge needle | with a syringe with a cannula |
| | | diameter of 25 gauge by means |
| | | of a universal testing machine; |
| | | see paragraph C) |
| 3 | High lubrication effect | Friction coefficients <0.2 |
| | | (tribometry cell measured by |
| | | means of a rheometer, see |
| | | paragraph C) |
| 4 | Anti-inflammatory effect | Reduction of the LPS-induced |
| | and relieving of the | swelling of the knee joint in |
| | swelling in the knee | the mouse model measured by |
| | joint in the mouse | means of caliper and gene |
| | model | expression of the inflammation |
| | | markers TBFa (see paragraph C) |

1. A therapeutic composition containing hydrogel particles, at least one hyaluronic acid component and at least one pharmaceutically acceptable excipient, in particular an aqueous fluid, wherein the hydrogel particles have at least one polyionic polymer component, which is covalently connected to at least one uncharged polymer component and/or a non-polymer crosslinker component by forming a network, and wherein the at least one polyionic polymer component has sulfate or sulfonate groups.

2. The therapeutic composition according to claim 1, wherein the at least one uncharged polymer component is selected from the group consisting of polyethylene glycol (PEG) component, poly(2-oxazoline) (POX) component, polyvinylpyrrolidone (PVP) component, polyvinyl alcohol (PVA) component, polyacrylamide (PAM) component and combinations thereof.

3. The therapeutic composition according to claim 1, wherein the at least one uncharged polymer component is a polyethylene glycol component, in particular a linear or multi-arm, in particular four- or eight-arm, polyethylene glycol component.

4. The therapeutic composition according to claim 1, wherein the at least one polyionic polymer component is a glycosaminoglycan component, in particular heparin or selectively desulfated heparin, in particular selectively N-desulfated heparin.

5. The therapeutic composition according to claim 1, wherein the at least one non-polymer crosslinker component is selected from the group consisting of ethylene diamine, propylene diamine (1,3-diaminopropane), butane-1,4-diamine, pentane-1,5-diamine (cadaverine), hexamethylene-1,6-diamine oxalic acid, malonic acid, succinic acid, glutaric acid, adipic acid, pimelic acid, N-(2-aminoethyl) maleimide and combinations thereof.

6. The therapeutic composition according to claim 1, wherein the hydrogel particles have a storage modulus of maximally 22 kPa, in particular 0.1 to 22 kPa, (in each case measured by hydrogel particles swollen in physiological saline solution (PBS).

7. The therapeutic composition according to claim 1, wherein the hydrogel particles have an average diameter of maximally 200 μm, in particular maximally 80 μm, (in each case measured by hydrogel particles swollen in physiological saline solution (PBS)).

8. The therapeutic composition according to claim 1, wherein the hydrogel particles have a sulfate or sulfonate group concentration of at least 0.1 mmol/l, in particular at least 10 mmol/l, in particular at least 100 mmol/l, in particular 0.1 to 800 mmol/l, in particular 10 to 800 mmol/l, in particular 20 to 500 mmol/l, in particular 20 to 200 mmol/l, in particular 50 to 200 mmol/l, (in each case measured in the volume of hydrogel particles swollen in physiological saline solution (PBS)).

9. The therapeutic composition according to claim 1, wherein the content of the hyaluronic acid component is 0.5 to 2.0% by weight, in particular 1.0 to 2.0% by weight (in each case based on total mass of the composition).

10. The therapeutic composition according to claim 1, wherein the therapeutic composition is an injectable therapeutic composition, in particular an injectable therapeutic composition, the injection force of which at an injection speed of 0.05 ml/s through a 25 G needle is maximally 10 N, in particular 2.7 to 3.5 N, in particular 2.7 to 3.5 N.

11. The therapeutic composition according to claim 1, wherein the friction coefficient of the composition is maximally 0.2, in particular 0.085 to 0.095, in each case at 10 revolutions per second.

12. The therapeutic composition according to claim 1, wherein the therapeutic composition is able to reduce the concentration of at least one free cytokine, in particular chemokine, in particular IL-8, in a biofluid, in particular in a synovial fluid, in particular in a model synovial fluid, in particular by more than 50%, in particular by 70 to 80%.

13. The therapeutic composition according to claim 1, wherein the uncharged polymer component and the polyionic polymer component are covalently connected to one another by means of at least one crosslinker component, in particular peptides, or directly, in particular by means of an amide bond, thiol-amine bond, disulfide bond or bio-orthogonal thioether bond, which can be obtained from thiol-maleimide, thiol-vinylsulfone or thiol-acrylate reaction.

14. The therapeutic composition according to claim 1, wherein the composition has a solids content of 4 to 25, in particular 5 to 20% by weight (in each case based on the total mass of the composition).

15. The therapeutic composition according to claim 1, wherein the composition additionally comprises at least one additive, in particular marine collagen, sorbitol, mannitol, platelet-rich plasma (PRP), polyphenols, S-allyl-cysteine, pentosan-polyphosphate-Na, and/or extracellular vesicles, containing curcuminoids.

16. A therapeutic composition for use in a method for treating arthritis, in particular osteoarthritis, rheumatoid arthritis, infectious arthritis, post-infectious arthritis, psoriatic arthritis or gouty arthritis, according to claim 1, in particular for the intra-articular injection into a joint of a human or animal patient.

17. A method for producing a therapeutic composition, in particular according to claim 1, comprising the following method steps:
a) providing at least one uncharged polymer, in particular a functionalized uncharged polymer, and/or at least one non-polymer functionalized crosslinker molecule, and at least one polyionic polymer having sulfate or sulfonate groups, at least one hyaluronic acid component and at least one pharmaceutically acceptable excipient as well as optionally further components,
b) crosslinking the uncharged polymer or non-polymer crosslinker molecule with the polyionic polymer by forming a network for obtaining a hydrogel, in particular of hydrogel particles, from at least one polyionic polymer component, which is covalently connected to at least one uncharged polymer component and/or a non-polymer crosslinker component,
c) mixing the hydrogel, in particular the hydrogel particles, with the at least one hyaluronic acid component and the at least one pharmaceutically acceptable excipient, and
d) obtaining the therapeutic composition.

18. The method for producing a therapeutic composition according to claim 17, wherein the uncharged polymer, in particular the functionalized uncharged polymer, and/or the at least one non-polymer functionalized crosslinker molecule has at least two functional groups, which are suitable to form a respective covalent bond to the polyionic polymer component, in particular selected from the group consisting of amino group, thiol group, maleimide group, vinylsulfone group, acrylate group, carboxyl group and combinations thereof.

19. The method for producing a therapeutic composition according to claim 17, wherein the polyionic polymer having at least one sulfate or sulfonate group has at least two functional groups selected from the group consisting of amino group, thiol group, maleimide group, vinylsulfone group, acrylate group, carboxyl group and combinations thereof.

20. The method for producing a therapeutic composition according to claim 17, wherein the hydrogel particles are formed in method step b) by means of the following additional method steps:
b1) fragmentation of the hydrogel, in particular by means of ultrasonic treatment with an ultrasonic homogenizer, to obtain hydrogel fragments and
b2) filtration of the hydrogel fragments to obtain hydrogel particles.

21. The method for producing a therapeutic composition according to claim 17, wherein the hydrogel particles are formed in method step b) by means of the following additional method steps:
b1′) producing an emulsion containing the at least one polyionic polymer as well as the at least one uncharged polymer and/or the at least one non-polymer crosslinker molecule,
b2′) cooling down the emulsion containing the least one polyionic polymer and the at least one uncharged polymer and/or the at least one non-polymer crosslinker molecule for obtaining cryogelated hydrogel particles,
b3′) lyophilization of the hydrogel particles,
b4′) purification of the lyophilized hydrogel particles,
wherein the at least one polyionic polymer as well as the at least one uncharged polymer and/or the at least one non-polymer crosslinker molecule are crosslinked during the method steps b1′) to b3′) to obtain the hydrogel.

22. The method for producing a therapeutic composition according to claim 17, wherein the hydrogel particles are formed in method step b) by means of the following additional method steps:
b1″) mixing the at least one polyionic polymer as well as the at least one uncharged polymer and/or the at least one non-polymer crosslinker molecule,
b2″) forming an emulsion by means of a microfluidic device and subsequent crosslinking to hydrogel particles, and
b3″) purifying the hydrogel particles,
wherein the at least one polyionic polymer and the at least one uncharged polymer and/or the at least one non-polymer crosslinker molecule are crosslinked during method step b2″) to obtain the hydrogel.

23. A therapeutic composition, which can be produced, in particular is produced, by means of a method according to claim 17.
